(19) United States
(12) Patent Application Publication
(10) Pub. No.: US 2023/0365911 A1
(43) Pub. Date: Nov. 16, 2023

(54) SYSTEM, METHOD, AND APPARATUS FOR MANUFACTURE OF ENGINEERED CELLS

(71) Applicant: Multiply Labs Inc., San Francisco, CA (US)

(72) Inventors: Ali RIZVI, San Francisco, CA (US); Andrew BECK, San Francisco, CA (US); Brigitte SCHMITTLEIN, San Francisco, CA (US); Dorothy SZYMKIEWICZ, San Francisco, CA (US); Federico PARIETTI, San Francisco, CA (US); Kameron CHAN, San Francisco, CA (US); Kelsey POOL, San Francisco, CA (US); Kenny HARSONO, San Francisco, CA (US); Nolan DICKEY, San Francisco, CA (US); Umberto SCARFOGLIERO, San Francisco, CA (US); Yasmine AINANE, San Francisco, CA (US)

(21) Appl. No.: 18/306,936

(22) Filed: Apr. 25, 2023

Related U.S. Application Data
(60) Provisional application No. 63/334,509, filed on Apr. 25, 2022.

(51) Int. Cl. C12M 1/42; C12M 1/12; C12M 1/34

(52) U.S. Cl. CPC C12M 35/02; C12M 37/02; C12M 41/26; C12M 41/40

(57) ABSTRACT

The present disclosure provides cartridges, pump assemblies, and other systems, methods, and apparatuses for manufacture of a cell therapy. A rigid cartridge includes rigid walls defining a rigid housing. An interior surface of a first rigid wall includes a first mating mechanism to engage a second mating mechanism of a docking device to removably coupled with the rigid cartridge. A rigid wall includes a planar upper surface including an aperture. The aperture is configured to receive and fixedly engage a corresponding connector.

### CROSS-REFERENCE TO RELATED APPLICATION

The present application claims priority to U.S. Provisional Patent Application No. 63/334,509, entitled “System, Method, and Apparatus for Manufacture of Engineered Cells,” filed Apr. 25, 2022, which is hereby incorporated by reference in its entirety for all purposes.

### TECHNICAL FIELD

The present invention is directed to systems, methods, and apparatuses for manufacture of engineered cells. More particularly, the present invention is directed to systems, methods, and apparatuses for manufacture of cell therapies.

### BACKGROUND

Traditionally, cell therapies and/or capsules are produced with labor-intensive processes. These conventional processes require not only a large number of manufacturing operators, but also the employment of highly skilled (and expensive) technicians. These constraints make it particularly difficult to manufacture cell therapies and/or capsules at an industrial scale.

Cell therapies are next-generation drugs where live cells are used to treat a subject. This is in contrast with traditional small-molecule and biologic drugs, where small or large molecules—but not whole living cells—are used to treat patients. Many of the most recent and promising innovations in medicine are represented by cell therapies in which the cells of a subject (either the patient or a donor) are extracted, genetically engineered in a lab, grown in an incubator, and finally infused in the patient in order to achieve a therapeutic effect. However, despite the life-saving effects of many cell therapies, there are significant bottlenecks to their widespread adoption. For instance, one obstacle is represented by the current limits in manufacturing capacity for cell therapies. Conventional cell therapy production processes are still largely labor-based and inefficient.

Traditionally, cell therapies are produced with labor-intensive processes. These conventional processes require not only a large number of manufacturing operators, but also the employment of highly skilled (and expensive) technicians. These constraints make it particularly difficult to manufacture cell therapies at an industrial scale. Cell therapy manufacturing processes are low-scale and labor-intensive because they were originally developed in the context of academic research. The original lab processes—which were developed to demonstrate the feasibility of cell therapies—were then hastily modified and retrofitted in order to fulfill regulatory requirements and achieve good manufacturing practices.

This conventional approach allowed drug manufacturers to bring to the market the first approved cell therapies. However, this labor-intensive, lab-oriented approach is unsuitable to achieve industrial scale. At their core, current cell manufacturing processes were designed to be manually completed by highly trained personnel—such as the researchers that conduct scientific experiments in an academic environment. Requiring this type of skillset becomes a disadvantage in an industrial setting. Cell manufacturing processes depend on highly trained, highly educated manual labor, and this makes them incompatible with the efficiency of mass-manufacturing industrial processes.

The dominant conventional approach to cell manufacturing is based on a set of separate individual pieces of manufacturing equipment placed on a clean room bench. This manufacturing process still looks exactly like a research laboratory, where all the machinery is manually operated and directly supervised by highly skilled operators. In order to execute the cell manufacturing processes, these skilled operators gown up, enter a clean room, and manually activate the machines. The operators also transfer the batch material from machine to machine, manually sample the batches to perform quality control testing, ensure that reagents are delivered to the cells, and ensure that waste material is removed. This labor-based conventional approach is very different from the organization of industrial-scale processes, where most tasks are autonomously executed by specialized machinery, which is supervised by ordinary manufacturing technicians (not engineers, nor scientists).

As such, the conventional labor-based approach to cell therapy manufacturing has at least three fundamental limits. First, the conventional approach is not scalable and not robust to operator variability. Because the conventional approach is extremely labor-intensive, cell therapy manufacturing is limited to small-scale applications. Increasing throughput beyond a few hundred products per year has proven extremely difficult, because such an effort would require hiring, training, retaining, and managing a large number of highly skilled, expensive operators. Moreover, labor-based processes are typically unable to reach industrial scale, and cell manufacturing is not an exception. This pronounced reliance of labor presents additional disadvantages, including the fact that—because of operator variability—the yield and the features of the finished cell therapy product are hard to predict and to control. This operator variability makes scaling the process of manufacturing cell therapy products even harder—particularly in terms of margins, in which a higher number of rejected batches increases the cost per batch.

Additionally, the conventional approach to manufacturing cell therapy products is inefficient. Since individual machines for the cell therapy manufacturing process are utilized in series (e.g., the machines are used one at a time, with a single batch manually moved from a piece of machinery to the next), when a machine is active all the others are idle. This results in a low utilization rate for all machines, since most of the machines are waiting for the batch to arrive, while a single machine is being used. The problem of a very low utilization rate is particularly evident for cell manufacturing processes, which are characterized by machines with markedly different cycle times. More specifically, systems like bioreactors process a single batch for weeks, while machines like thawing and freezing systems are only used for a few hours on a single batch. This results in utilization rates that are even lowed for the faster machines—because the slower machines are the bottleneck and limit the rate of the rest of the serial process.

Finally, the conventional approach to manufacturing cell therapy products has low throughput. Because the process is managed and executed by human operators, only one batch can be produced at any given time on a serial production line. For instance, if two batches were manufactured at the same time on the same production line, in fact, there would be high risk of cross-contamination or of mix-up errors by the operators. Since all the serial machines are used for just one product at a time, the resulting throughput of the production line is extremely low. As a reference, typically a cell therapy product takes two to three weeks to be manufactured. This means that, in order to avoid mix-ups, a whole production line must be reserved for a single product for about half of a month—a rate that is incompatible with industrial scale. Because of this temporal constraint, a whole manufacturing suite (typically consisting of about 1,000 square feet of clean room space) must be reserved for a single serial production line. Therefore, the only way to increase throughput via this conventional approach is by creating facilities with multiple independent suites that replicate the same process. However, each suite can only handle one product at a time, occupies significant clean room space, and is entirely operated by skilled labor. As such, this conventional approach is not scalable, and not suitable to manufacture more than a few hundreds of cell therapies per year—with very high production costs.

One solution to this conventional approach are closed system cell therapy machines that have been developed to attempt to address the shortcomings of the traditional approach. However, even this solution is still labor-intensive and inadequate to reach industrial scale. For instance, this solution can be described as an end-to-end serial system that is contained into a single machine. Different parts of the same machine perform the different steps of the production process. In other words, a single piece of equipment contains all the sub-systems that are needed to perform the cell manufacturing process. An intricate set of tubes connects all of these systems, so that the cell therapy product (which is typically in liquid form) can be transferred from one sub-system to the next without being exposed to the external environment, which provides the closed system.

However, these end-to-end, closed systems are sold as a unique piece of machinery. As such, the machinery cannot be modified by the buyer: once a system is bought, the buyer is constrained to run the exact process for which that machine was designed. Additionally, the machinery still needs to be operated by a highly skilled technician, who needs to perform a complicated set of actions to set up, monitor, and manage the manufacturing process. More specifically, highly trained operators set up the intricate network of tubes that is required by each batch. These operators are also tasked with opening and closing the valves that regulate the flow of material from one part of the system to the next. Furthermore, technicians also manually sample the batch, whenever testing is needed for quality control.

As such, this prior closed system solution suffers disadvantages, in that the closed system solution is overcomplicated. Setting up dozens of tubes, liquid reservoir bags, and reagents requires highly trained labor. This setting up process also takes a long time—even for a skilled technician—to set up, operate, and supervise the machinery. This results in the need for a number of operators that increases proportionally to the number of production system—making it impossible to achieve industrial scale and contain manufacturing costs.

Furthermore, the prior closed system solution is inefficient. Since the architecture of the closed system is still serial, this approach suffers of the same efficiency constraints as the dominant (bench-based) approach. At any given time, most of the subsystems inside of the end-to-end machine are unused. This happens because only one system can be used at a time—this is a serial production line with the hard limit of a single product per production run. Moreover, since some parts of the process are particularly slow (for example, the expansion of the cells into a bioreactor), the “aster subsystems are characterized by an even lower utilization rate than the slower subsystems of the machinery.

Additionally, this closed system lacks design flexibility. This inflexibility drawback is typical of closed systems that are built specifically to execute a particular process. Once the machinery is bought, it is not possible to replace an outdated subsystem with a better one (for example, a subsystem that performs a task better, or with a higher throughput). Any modification to the original closed system machinery requires massive engineering and retooling costs, comparable to building a whole new end-to-end system from scratch. This lack of flexibility is particularly disadvantageous in the case of cell therapy manufacturing—where processes are often tuned and improvement at all stages of clinical development.

Moreover, since each closed system is end-to-end and can only manufacture a single product at a time, the only way to increase throughput is to buy more of these closed systems. This in turn worsens the above-mentioned complexity and underutilization problems. In other words, deploying more complex systems increases the need for skilled operators, which in turn increases the cost of manufacturing. Since each machine is largely underutilized (only one subsystem is active at any given time), chronic underutilization also characterizes a facility that is equipped with multiple end-to-end systems. Furthermore, conventional docking station designs does lend themselves to application in cell therapy manufacturing. For instance, conventional docking stations do not include passive compliance and passive damping systems.

Additionally, a major problem of labor-based cell manufacturing processes is that human operators need to sample each batch manually. In cell manufacturing processes, sterility must be always ensured. This is particularly important, because cell therapies cannot be sterilized at the end of the manufacturing process (that would kill the cells). At the same time, guaranteeing the quality of cell manufacturing processes requires a large number of quality control steps. And, in order to perform quality control tests, the cell therapy products must be frequently sampled (i.e., a part of the product must be removed from the batch, while ensuring the sterility of both the sample and the product). In conventional cell manufacturing processes, sampling tasks are executed by human operators.

One disadvantage of this conventional approach to sampling is that human operators are a significant potential source of contamination for cell therapy products. Every time a batch is sampled manually, there is a high risk of contamination because the operator must manually remove a part of the liquid containing the cell product. Even semi-automated sampling procedures, where an operator activates a system that performs the sampling task, present significant risk of contamination due to requiring the presence of a human technicians in close proximity to the process.

Another critical issue is that sampling procedures are performed extremely frequently in cell manufacturing processes. Cell therapy products are sometimes sampled multiple times during a single day. Since cell manufacturing processes have a long completion time (most require more than a week, and many can take up to fifteen to twenty days), manual sampling is repeated dozens of times for every single batch. Repeating risky sampling procedures with this extreme frequency greatly increases the risk of contamination.

Given the above background, there is a need in the art for improved systems, methods, and apparatuses for facilitating an improved manufacture of cell therapies that addresses these dilemmas.

The information disclosed in this background section is only for enhancement of understanding of the general background of the invention and should not be taken as an acknowledgement or any form of suggestion that this information forms the prior art already known to a person skilled in the art.

Given the above-background, what is needed in the art are improved systems, methods, and apparatuses for manufacture of capsules and/or cell therapies.

### SUMMARY

The present disclosure addresses the shortcomings disclosed above by providing systems, methods, and apparatuses for manufacture of cell therapies.

In various embodiments, the present disclosure provides an apparatus for manufacture of an engineered cells. The apparatus includes a rigid cartridge. The rigid cartridge includes a plurality of rigid walls that define a fixed interior. Moreover, the plurality of rigid walls includes a plurality of rigid side walls. At least one interior surface of a rigid side wall in the plurality of side walls includes a first mating mechanism that configured to engage a corresponding second mating mechanism of a docking device. Accordingly, the first mating mechanism and the corresponding second mating mechanism allow for removably coupling the rigid cartridge to the docking device when the first mating mechanism interfaces with the corresponding second mating mechanism to restrict a movement of the rigid cartridge to at least one degree of freedom. The rigid cartridge includes a substantially planar upper rigid wall that connected to an upper edge portion of each rigid side wall in the plurality of rigid side walls. Furthermore, the substantially planar upper rigid wall includes one or more apertures. Each respective aperture in the one or more apertures is configured to receive and fixedly engage a corresponding connector in one or more connectors. Moreover, each respective connector in the one or more connectors interfaces with a corresponding portion of a fixed interior of the rigid cartridge.

In some embodiments, the at least one interior surface is in the pair of opposing interior surfaces of two rigid side walls in the plurality of rigid side walls that each fourth second mating mechanism of a soft body bioreactor accommodated by the rigid cartridge, which allows for removably coupling a pair of opposing end portions of the soft body bioreactor with the rigid cartridge.

In some embodiments, the one or more connectors includes a fluidic connector and/or an electrical connector.

In some embodiments, the electrical connector includes a pressure control mechanism, a pH sensor, a dissolved oxygen sensor, a temperature sensor, a flow rate sensor, a mass sensor, or a combination thereof.

In some embodiments, the fluidic connector includes a pressure control mechanism, an inlet port, a sampling port, an outlet port, or a combination thereof.

In some embodiments, the fluidic connector is in communication with a manifold configured to generate a pressure gradient between a portion of the rigid cartridge and an interior of the manifold.

In some embodiments, the fluidic connector includes a valve configured to control a flow of a fluid through a corresponding fluidic port.

In some embodiments, a first internal diameter at an upper end portion of a respective connector is less than a second internal diameter at a lower end portion of the respective connector.

In some embodiments, at least one rigid wall in the plurality of rigid walls further includes a gate mechanism configured to provide access to a fixed internal cavity of the rigid housing.

In some embodiments, each mating mechanism protrudes from the substantially upper planar surface of the at least one rigid wall of the rigid housing.

In some embodiments, the apparatus further includes a corresponding cap for each respective connector in the plurality of connectors, in which the corresponding cap encompasses the respective connector.

In some embodiments, the rigid cartridge further includes a lower end portion that includes an opening configured to accommodate a port of fluidic container.

In some embodiments, the first mating mechanism includes one or more magnets, one or more fasteners, one or more snap-fittings, one or more press-fittings, one or more adhesives, one or more grooves, one or more holes, one or more openings, one or more protrusions, one or more pins, one or more wedges, one or more indica, one or more snap mechanisms, one or more ramps, one or more springs, or a combination thereof.

In some embodiments, the first degree of freedom is parallel or substantially parallel to gravity.

In some embodiments, the first degree of freedom is not parallel or substantially parallel to gravity.

In some embodiments, an interior angle formed between a substantially planar upper rigid wall and each rigid sidewall in the plurality of rigid side walls is greater than or equal to 90 degrees.

In some embodiments, an exterior surface of the rigid cartridge includes one or more identifiers indica configured to uniquely identify the rigid cartridge.

In some embodiments, an exterior surface of the rigid cartridge includes one or more orientation indica configured to identify an orientation of the rigid cartridge.

In some embodiments, the corresponding second locking mechanism includes a rotating fastener configured to pivot between a first position that restricts a movement of the rigid cartridge and a second position that allows for movement of the rigid cartridge.

In some embodiments, the first locking mechanism and the corresponding second locking mechanism collectively form an undercarriage of the apparatus.

In various embodiments, the present disclosure provides a computer system for manufacture of a cell therapy.

In various embodiments, the present disclosure provides a non-transitory computer readable storage medium storing one or more programs, the one or more programs including instructions, which when executed by an electronic device with one or more processors and a memory cause the electronic device to execute a method for manufacture of a cell therapy.

In various embodiments, the present disclosure provides a method for manufacture of a cell therapy.

### BRIEF DESCRIPTION OF THE DRAWINGS

The Figures collectively illustrate systems, methods, and apparatuses for manufacture of capsules and/or cell therapies, in accordance with some embodiments of the present disclosure.

FIG. 1 illustrates a block diagram illustrating an embodiment of a system for manufacture of engineered cells, in accordance with embodiments of the present disclosure;

FIG. 2 illustrates various modules and/or components of a computer system, in accordance with an exemplary embodiment of the present disclosure;

FIG. 3 is a perspective view of a modular clean room biological foundry system including a plurality of instruments, in accordance with an embodiment of the present disclosure;

FIG. 4 is a top view of a modular clean room biological foundry system including a plurality of instruments, in accordance with an embodiment of the present disclosure;

FIG. 5 is a perspective view of another modular clean room biological foundry system including a plurality of instruments, in accordance with an embodiment of the present disclosure;

FIG. 6A illustrates a medium distribution system of a modular clean room biological foundry system, in accordance with an embodiment of the present disclosure;

FIG. 6B illustrates another medium distribution system of a modular clean room biological foundry system, in accordance with an embodiment of the present disclosure;

FIG. 6C illustrates yet another medium distribution system of a modular clean room biological foundry system, in accordance with an embodiment of the present disclosure;

FIG. 6D illustrates yet another medium distribution system of a modular clean room biological foundry system, in accordance with an embodiment of the present disclosure;

FIG. 6E illustrates a side view of a connector of a rigid cartridge, in accordance with an embodiment of the present disclosure;

FIG. 6F illustrates a perspective view of the connector of FIG. 6E;

FIG. 7A illustrates a top perspective of a rigid cartridge, in accordance with an embodiment of the present disclosure;

FIG. 7B illustrates a bottom perspective of another rigid cartridge, in accordance with an embodiment of the present disclosure;

FIG. 7C illustrates a top perspective of yet another a rigid cartridge, in accordance with an embodiment of the present disclosure;

FIG. 7D illustrates a top perspective of a docker device, in accordance with an embodiment of the present disclosure;

FIG. 7E illustrates a side cross-sectional view of the docker device of FIG. 7D;

FIG. 7F illustrates a rear perspective of the docker device of FIG. 7D;

FIG. 7G illustrates a gripping device, in accordance with an embodiment of the present disclosure;

FIG. 7H illustrates an apparatus including a rigid cartridge, a docker device, and a gripping device, in accordance with an embodiment of the present disclosure;

FIG. 7I illustrates a cross-sectional view of the apparatus of FIG. 7H;

FIG. 8A illustrates an apparatus including a rigid cartridge and a docker device, in accordance with an embodiment of the present disclosure;

FIG. 8B illustrates a cross-sectional view of the apparatus of FIG. 8A, in accordance with an embodiment of the present disclosure;

FIG. 8C illustrates a partially exploded view of the apparatus of FIG. 8A;

FIG. 9A illustrates a rear perspective of a rigid cartridge, in which a gate mechanism is in a closed configuration, in accordance with an embodiment of the present disclosure;

FIG. 9B illustrates a front perspective of a rigid cartridge, in which a gate mechanism is in an opened configuration, in accordance with an embodiment of the present disclosure;

FIG. 9C illustrates a cross-sectional view of the rigid cartridge of FIG. 9A;

FIG. 10A illustrates a mating mechanism of a docking device, in accordance with an embodiment of the present disclosure;

FIG. 10B illustrates a cross-sectional view of the docking device of FIG. 10A;

FIG. 10C illustrates a top perspective view of a latch portion of a mating mechanism of a docking device, in accordance with an embodiment of the present disclosure;

FIG. 10D illustrates a bottom perspective view of the latch portion of FIG. 10C;

FIG. 10E illustrates a mating mechanism of a docking device in a locked position and an unlocked position, in accordance with an embodiment of the present disclosure;

FIG. 11A illustrates a rear perspective of a rigid cartridge, in accordance with an embodiment of the present disclosure;

FIG. 11B illustrates a front perspective of the rigid of FIG. 11A;

FIG. 11C illustrates another rear perspective of a rigid cartridge, in accordance with an embodiment of the present disclosure;

FIG. 11D illustrates a front perspective of the rigid of FIG. 11C;

FIG. 11E illustrates another rear perspective of a rigid cartridge, in accordance with an embodiment of the present disclosure;

FIG. 11F illustrates a front perspective of the rigid cartridge of FIG. 11E;

FIG. 11G illustrates a cartridge of an apparatus, in accordance with an embodiment of the present disclosure;

FIG. 11H illustrates another cartridge of an apparatus, in accordance with an embodiment of the present disclosure;

FIG. 11I illustrates yet another cartridge of an apparatus, in accordance with an embodiment of the present disclosure;

FIG. 12A illustrates a cartridge of an apparatus, in accordance with an embodiment of the present disclosure;

FIG. 12B illustrates another cartridge of an apparatus, in accordance with an embodiment of the present disclosure;

FIG. 12C illustrates yet another cartridge of an apparatus, in accordance with an embodiment of the present disclosure;

FIG. 12D illustrates yet another cartridge of an apparatus, in accordance with an embodiment of the present disclosure;

FIG. 13 illustrates a perspective of a rigid cartridge, in accordance with an embodiment of the present disclosure;

FIG. 14A illustrates a perspective of a rigid cartridge, in accordance with an embodiment of the present disclosure;

FIG. 14B illustrates a perspective of another rigid cartridge, in accordance with an embodiment of the present disclosure;

FIG. 14C illustrates a front of the rigid cartridge of FIG. 14A, in accordance with an embodiment of the present disclosure;

FIG. 15 illustrates a perspective of a rigid cartridge, in accordance with an embodiment of the present disclosure;

FIG. 16A illustrates a tube portion of a manifold of an apparatus, in accordance with an embodiment of the present disclosure;

FIG. 16B illustrates another tube portion of a manifold of an apparatus, in accordance with an embodiment of the present disclosure; and

FIG. 16C illustrates yet another tube portion of a manifold of an apparatus, in accordance with an embodiment of the present disclosure.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

For convenience in explanation and accurate definition in the appended claims, the terms “upper,” “lower,” “up,” “down,” “upwards,” “downwards,” “inner,” “outer,” “inside,” “outside,” “inwardly,” “outwardly,” “interior,” “exterior,” “front,” “rear,” “back,” “forwards,” and “backwards,” are used to describe features of the exemplary embodiments with reference to the positions of such features as displayed in the figures.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

Furthermore, when a reference number is given an “i^{th}” denotation, the reference number refers to a generic component, set, or embodiment. For instance, a client device termed “client device i” refers to the i^{th} client device in a plurality of client devices (e.g., a client device 100-i in a plurality of client devices 100).

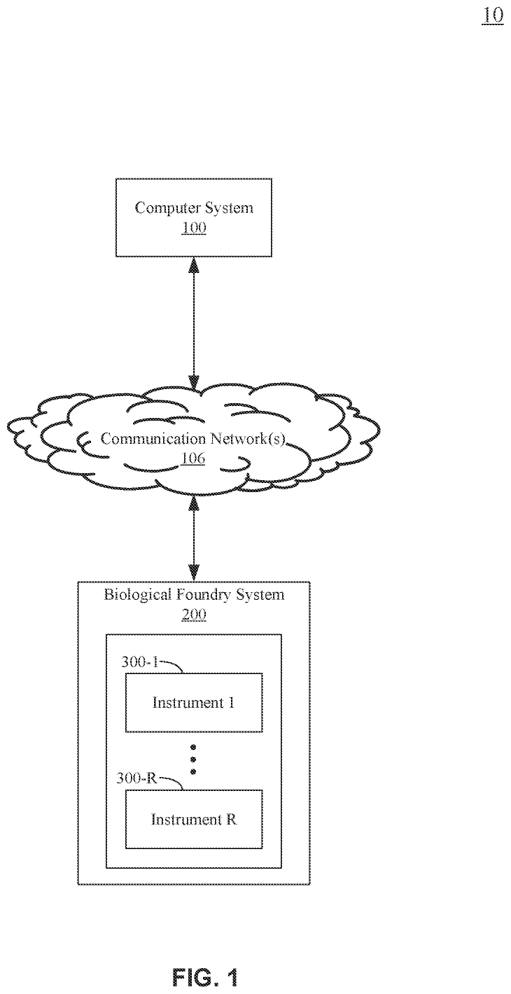

In the present disclosure, unless expressly stated otherwise, descriptions of devices and systems will include implementations of one or more computers. For instance, and for purposes of illustration in FIG. 1, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 106 of FIG. 1). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

An aspect of the present disclosure is directed to providing systems, methods, and apparatuses for facilitating automated modular manufacture of cell therapies.

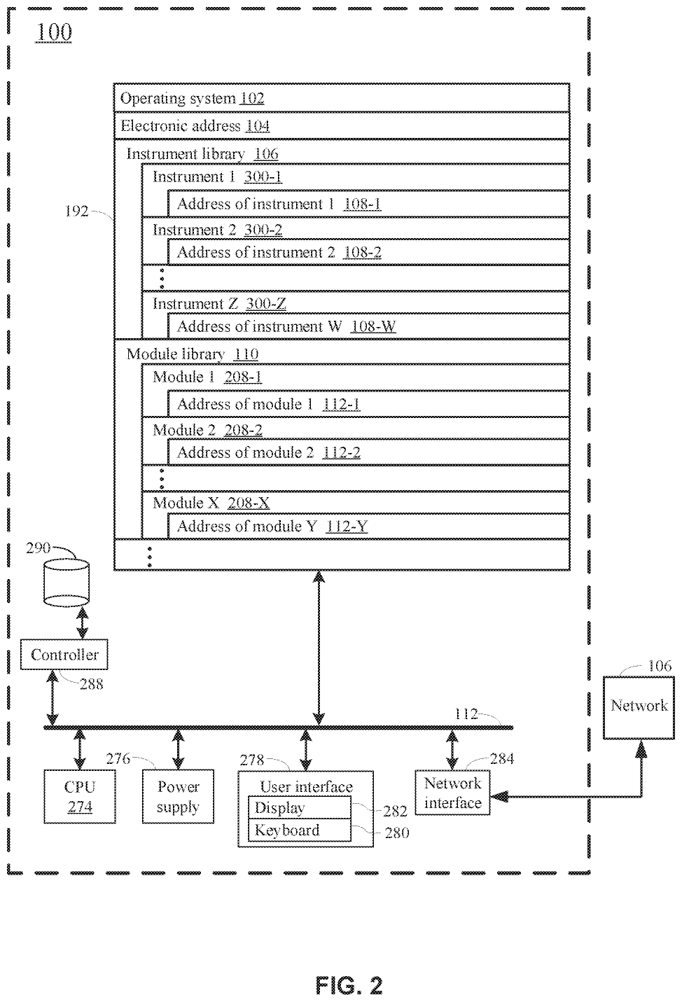

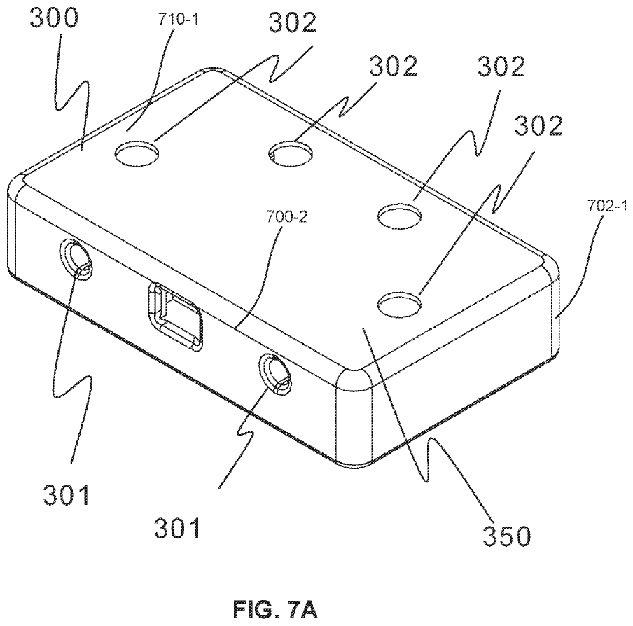

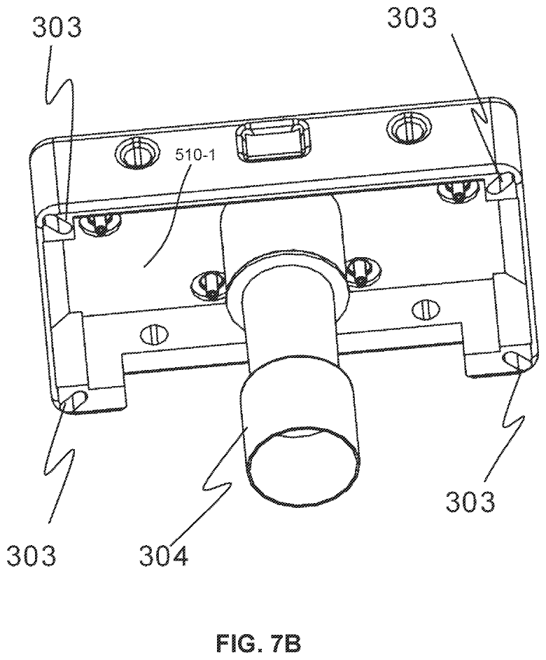

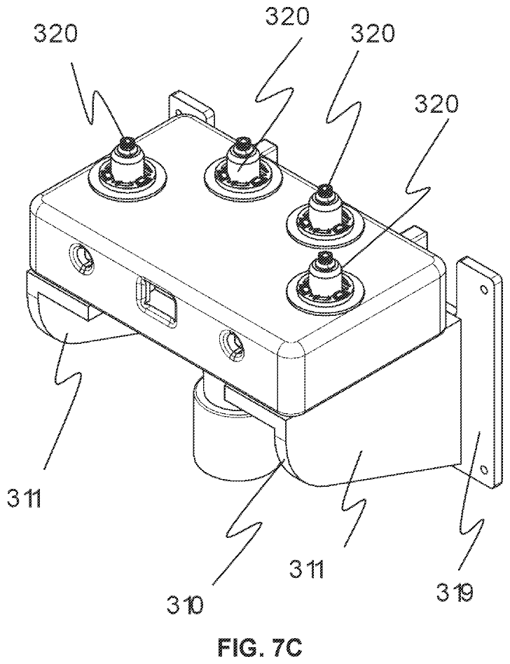

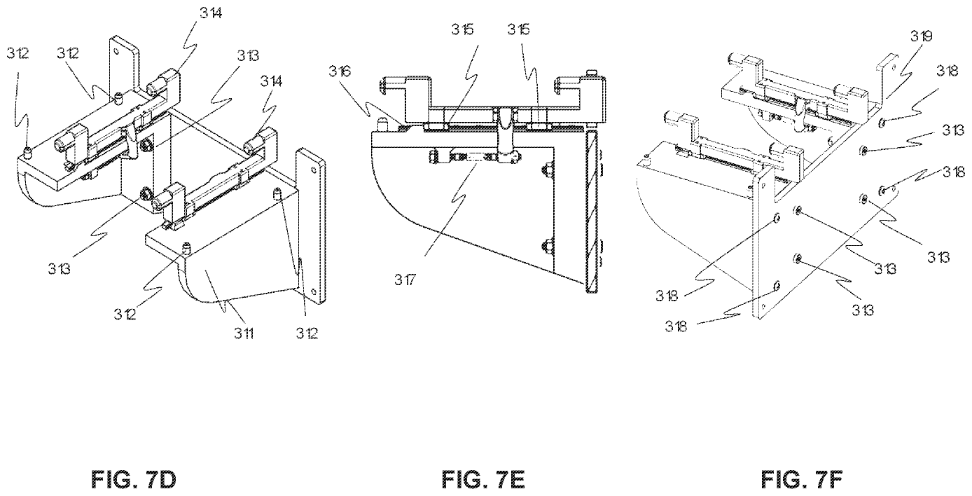

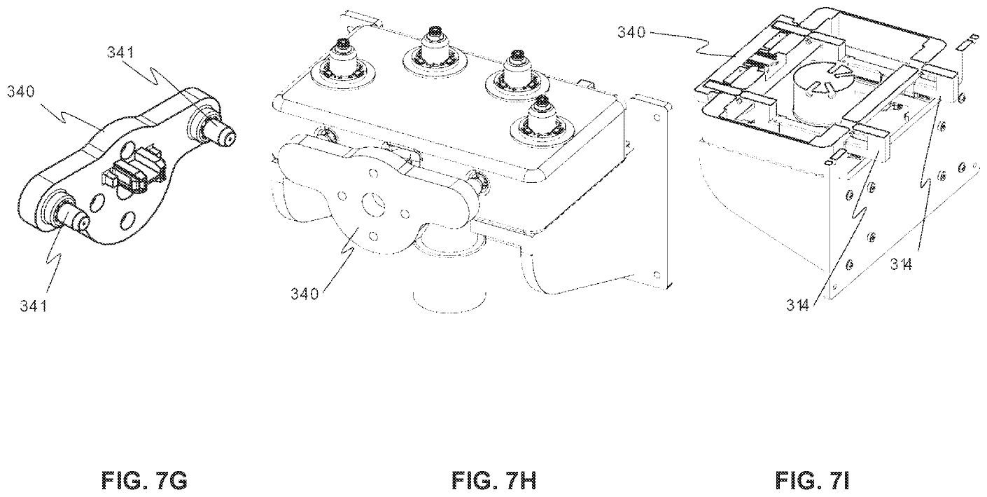

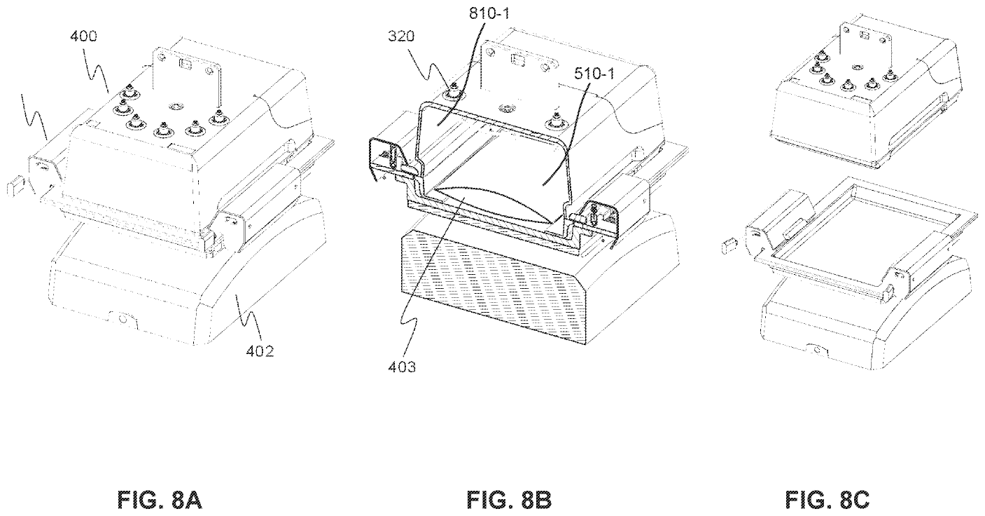

A detailed description of an exemplary system 10 for implementing the automated modular production of cellular engineering targets (e.g., cell therapies) at a biological foundry 200 is described in conjunction with FIG. 1 and FIG. 2. As such, FIG. 1 and FIG. 2 collectively illustrate an exemplary topology of the system 10. In the topology, there is a computer system 100 for generating a workflow that produces a plurality of cellular engineering targets, and providing scheduling of a plurality of instruments (e.g., first instrument 300-1, . . . , instrument 300-R of FIG. 1; instrument 300-1 of FIG. 7, instrument 300-2 of FIG. 8, instrument 300) in correlation with a corresponding plurality of biological foundry operations, and oversight of the manufacture of the plurality of cellular engineering targets at the modular biological foundry system.

In some embodiments, each cellular engineering, in the context of biological engineering at a modular biological foundry system, is one of the objectives of a research and development project that defines the desired biological trait to be achieved. The cellular engineering target can be either quantitative or qualitative. For example, in one embodiment, a cellular engineering target(s) can be a genetic configuration for a biosynthetic pathway that produces more compound of interest than a current level. In another embodiment, the cellular engineering target(s) is a genetic configuration for a microbial host that has a tolerance to an inhibitor over X mg/L.

In some embodiments, each cellular engineering target includes modified immune cells or precursors thereof, such as modified T cells, including a chimeric antigen receptor (CAR). Thus, in some embodiments, the immune cell is genetically modified at a modular biological foundry system to express the CAR. In some embodiments, CARs include an antigen binding domain, a transmembrane domain, a hinge domain, and an intracellular signaling domain.

In some embodiments, the antigen binding domain is operably linked to another domain of the CAR, such as the transmembrane domain or the intracellular domain, for expression in the cellular engineering target. In some embodiments, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains, any of the intracellular domains or cytoplasmic domains, or any of the other domains that may be included in a CAR. In some embodiments, a cellular engineering target CAR of the present disclosure includes a spacer domain. In some embodiments, each of the antigen binding domain, transmembrane domain, and intracellular domain is separated by a linker.

In the present disclosure, the cellular engineering targets generally include mammalian cells, and typically include human cells. In some embodiments, the cellular engineering target is derived from the blood, bone marrow, lymph, or lymphoid organs. In some embodiments, the cellular engineering targets includes cells of the immune system, such as cells of innate or adaptive immunity, e.g., myeloid or lymphoid cells, including lymphocytes, typically T cells and/or NK cells. In some embodiments, the cellular engineering targets include stem cells, such as multipotent and pluripotent stem cells, including induced pluripotent stem cells (iPSCs). The cellular engineering targets typically are primary cells, such as those isolated directly from a subject and/or isolated from a subject and frozen. In some embodiments, the cellular engineering targets include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, degree of differentiation, or a combination thereof. With reference to the subject to be treated, the cellular engineering targets be allogeneic and/or autologous. In some embodiments, the modular biological foundry system facilitates manufacturing the cellular engineering targets by isolating cells from the subject, preparing the cells, processing the cells, culturing the cells, engineering the cells, and re-introducing the cells into the same subject, before or after cryopreservation. In some embodiments, one or more of these steps is performed at modules 208 of modular biological foundry system 200 by utilizing an apparatus including rigid cartridge 300, docking device, gripping device, medium distribution system, or a combination thereof.

In some embodiments, among the sub-types and subpopulations of T cells and/or of CD4+ and/or of CD8+ T cells of the cellular engineering targets are naive T (T.sub.N) cells, effector T cells (T.sub.EFF), memory T cells and sub-types thereof, such as stem cell memory T (T.sub.SCM), central memory T (T.sub.CM), effector memory T (T.sub.EM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cellular engineering targets are natural killer (NK) cells. In some embodiments, the cellular engineering targets are monocytes or granulocytes, e.g., myeloid cells, macrophages, neutrophils, dendritic cells, mast cells, eosinophils, and/or basophils.

Accordingly, the present disclosure provides systems and methods for producing or generating a cellular engineering target that is a modified immune cell or precursor thereof (e.g., a T cell) of the invention for tumor immunotherapy, e.g., adoptive immunotherapy. The cellular engineering targets generally are engineered by introducing one or more nucleic acids encoding a subject CAR, dominant negative receptor and/or switch receptor, and/or bispecific antibody, and/or combinations thereof.

In some embodiments, one or more nucleic acids encoding the subject CAR, dominant negative receptor and/or switch receptor, and/or bispecific antibody is introduced into a cell by an expression vector. Expression vectors including a nucleic acid sequence encoding a subject CAR, dominant negative receptor and/or switch receptor, and/or bispecific antibody, and/or combinations thereof, of the present disclosure are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR, dominant negative receptor and/or switch receptor, and/or bispecific antibody, and/or combinations thereof, in the host cell. In some embodiments, the adenovirus genome is a 36 kb, linear, double stranded DNA, where a foreign DNA sequence (e.g., a nucleic acid encoding a subject CAR, dominant negative receptor and/or switch receptor, and/or bispecific antibody, and/or combinations thereof) may be inserted to substitute large pieces of adenoviral DNA in order to make the expression vector of the present invention. Additional details and information can be found at Danthinne et al., 2002, Gene Therapy, 7(20, pg. 1707, which is hereby incorporated by reference in its entirety.

Another expression vector is based on an adeno associated virus, which takes advantage of the adenovirus coupled systems. This AAV expression vector has a high frequency of integration into the host genome. Moreover, this AAV expression can infect non-dividing cells, thus making it useful for delivery of genes into mammalian cells, for example, in tissue cultures or in vivo. The AAV vector has a broad host range for infectivity.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR, dominant negative receptor and/or switch receptor, and/or bispecific antibody, and/or combinations thereof) into the viral genome at certain locations to produce a virus that is replication defective. Though the retrovirus vectors are able to infect a broad variety of cell types, integration and stable expression of the subject CAR, dominant negative receptor and/or switch receptor, and/or bispecific antibody, and/or combinations thereof, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR, dominant negative receptor and/or switch receptor, and/or bispecific antibody, and/or combinations thereof.

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistic, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell. In some embodiments, one or more of these steps is performed at modules 208 of modular biological foundry system 200 by utilizing rigid cartridge 300, docking device, gripping device, medium distribution system, or a combination thereof.

The present invention also provides genetically engineered cells which include and stably express a subject CAR, dominant negative receptor and/or switch receptor, and/or bispecific antibody, and/or combinations thereof, of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In one embodiment, the genetically engineered cells are autologous cells.

In some embodiments, modified cells (e.g., including a subject CAR, dominant negative receptor and/or switch receptor, and/or expresses and secretes a bispecific antibody, and/or combinations thereof) is produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR, dominant negative receptor and/or switch receptor, and/or bispecific antibody, and/or combinations thereof, of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, Sambrook et al, 2001, “Molecular Cloning: A Laboratory Manual,” Cold Spring Harbor Laboratory, print, which is hereby incorporated by reference in its entirety. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

In some embodiments, lipids suitable for use in the manufacture of a cellular engineering target at a modular biological foundry system is obtained from commercial sources. Stock solutions of lipids in chloroform or chloroform/methanol can be stored at about −20 degrees C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Compositions that have different structures in solution than the normal vesicular structure are also encompassed. For example, the lipids may assume a micellar structure or merely exist as non-uniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the invention. In some embodiments, one or more of these assays is performed at modules 208 of modular biological foundry system 200 by utilizing rigid cartridge 300, docking device, gripping device, medium distribution system, or a combination thereof.

In one embodiment, the nucleic acids introduced into the host cell are RNA. In another embodiment, the RNA is mRNA that includes in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

In some embodiments, chemical structures that have the ability to promote stability and/or translation efficiency of the RNA are used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art.

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a subject CAR, dominant negative receptor and/or switch receptor, and/or bispecific antibody, and/or combinations thereof, of the present disclosure will be RNA, e.g., in vitro synthesized RNA. Methods for in vitro synthesis of RNA are known in the art; any known method can be used to synthesize RNA including a sequence encoding a subject CAR, dominant negative receptor and/or switch receptor, and/or bispecific antibody, and/or combinations thereof. Methods for introducing RNA into a host cell are known in the art. Introducing RNA including a nucleotide sequence encoding a subject CAR, dominant negative receptor and/or switch receptor, and/or bispecific antibody, and/or combinations thereof, into a host cell can be carried out in vitro or ex vivo or in vivo. For example, a host cell (e.g., an NK cell, a cytotoxic T lymphocyte, etc.) can be electroporated in vitro or ex vivo with RNA including a nucleotide sequence encoding a subject CAR, dominant negative receptor and/or switch receptor, and/or bispecific antibody, and/or combinations thereof.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. The various parameters including electric field strength required for electroporation of any known cell type are generally known in the relevant research literature as well as numerous patents and applications in the field. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the subject CAR, dominant negative receptor and/or switch receptor, and/or bispecific antibody, and/or combinations thereof. In some embodiments, the cells (e.g. T cells) can be incubated or cultivated prior to, during or subsequent to the introduction of the nucleic acid molecule encoding the subject CAR, dominant negative receptor and/or switch receptor, and/or bispecific antibody, and/or combinations thereof, such as prior to, during or subsequent to the transduction of the cells with a viral vector (e.g. lentiviral vector) encoding the subject CAR, dominant negative receptor and/or switch receptor, and/or bispecific antibody, and/or combinations thereof. In some embodiments, the method includes activating or stimulating cells with a stimulating or activating agent (e.g., anti-CD3/anti-CD28 antibodies) prior to introducing the nucleic acid molecule encoding the subject CAR, dominant negative receptor and/or switch receptor, and/or bispecific antibody, and/or combinations thereof. In some embodiments, one or more of these steps is performed at modules 208 of modular biological foundry system 200 by utilizing rigid cartridge 300, docking device, gripping device, medium distribution system, or a combination thereof.

In some embodiments, where the nucleic acid sequences encoding the subject CAR, dominant negative receptor and/or switch receptor, and/or bispecific antibody, and/or combinations thereof, of the present invention reside on one or more separate nucleic acid sequences, the order of introducing each of the one or more nucleic acid sequences may vary. For example, a nucleic acid sequence encoding a subject CAR and dominant negative receptor and/or switch receptor may first be introduced into the host cell, followed by introduction of a nucleic acid sequence encoding a subject bispecific antibody. For example, a nucleic acid sequence encoding a subject bispecific antibody may first be introduced into the host cell, followed by introduction of a nucleic acid sequence encoding a subject CAR and dominant negative receptor and/or switch receptor. In some embodiments, each of the one or more nucleic acid sequences are introduced into the host cell simultaneously. Those of skill in the art will be able to determine the order in which each of the one or more nucleic acid sequences are introduced into the host cell.

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some embodiments, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some embodiments, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, one or more of these steps is performed at modules 208 of modular biological foundry system 200 by utilizing rigid cartridge 300, docking device, gripping device, medium distribution system, or a combination thereof.

In some examples, cells from the circulating blood of a subject are obtained, e.g., by apheresis or leukapheresis. The samples, in some embodiments, contain lymphocytes, including T cells, monocytes, granulocytes, B cells, other nucleated white blood cells, red blood cells, and/or platelets, and, in some embodiments, contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient. In some embodiments, one or more of these steps is performed at modules 208 of modular biological foundry system 200 by utilizing rigid cartridge 300, docking device, gripping device, medium distribution system, or a combination thereof.

In one embodiment, immune cells are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample are removed and the cells directly resuspended in culture media. In some embodiments, one or more of these steps is performed at modules 208 of modular biological foundry system 200 by utilizing rigid cartridge 300, docking device, gripping device, medium distribution system, or a combination thereof.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, one or more of these steps is performed at modules 208 of modular biological foundry system 200 by utilizing cartridge 300, docking device, gripping device, medium distribution system, or a combination thereof.

Such separation steps can be based on positive selection, in which the cells having bound the reagents are retained for further use, and/or negative selection, in which the cells having not bound to the antibody or binding partner are retained. In some examples, both fractions are retained for further use. In some embodiments, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker.sup.high) of one or more particular markers, such as surface markers, or that are negative for (marker.sup.-) or express relatively low levels (marker.sup.low) of one or more markers. For example, in some embodiments, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some embodiments, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which, in some embodiments, is particularly robust in such sub-populations. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L− CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some embodiments, it is based on negative selection for cells expressing or highly expressing CD45RA and/or granzyme B. In some embodiments, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections, in some embodiments, are carried out simultaneously and in other aspects are carried out sequentially, in either order. In some embodiments, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some embodiments, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further include the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In some embodiments, one or more of these steps is performed at modules 208 of modular biological foundry system 200 by utilizing cartridge 300, docking device, gripping device, medium distribution system, or a combination thereof.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques. In some embodiments, one or more of these steps is performed at modules 208 of modular biological foundry system 200 by utilizing cartridge 300, docking device, gripping device, medium distribution system, or a combination thereof.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample including an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immuno-adherence or flow cytometry that uses a cocktail of monoclonal antibodies directed to cell surface markers present on the cells negatively selected. For example, to enrich for CD4.sup.+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. In some embodiments, one or more of these steps is performed at modules 208 of modular biological foundry system 200 by utilizing cartridge 300, docking device, gripping device, medium distribution system, or a combination thereof.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80 degrees C. at a rate of 1 degrees C. per minute and stored in the vapor phase of a liquid nitrogen storage tank. Other methods of controlled freezing may be used as well as uncontrolled freezing immediately at −20 degrees C. or in liquid nitrogen. In some embodiments, one or more of these steps is performed at modules 208 of modular biological foundry system 200 by utilizing rigid cartridge 300, docking device, gripping device, medium distribution system, or a combination thereof.

In one embodiment, the population of T cells includes cells such as peripheral blood mononuclear cells, cord blood cells, a purified population of T cells, and a T cell line. In another embodiment, peripheral blood mononuclear cells include the population of T cells. In yet another embodiment, purified T cells include the population of T cells.

In certain embodiments, T regulatory cells (Tregs) is isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. In some embodiments, one or more of these steps is performed at modules 208 of modular biological foundry system 200 by utilizing rigid cartridge 300, docking device, gripping device, medium distribution system, or a combination thereof.

Whether prior to or after modification of cells to express a subject CAR, dominant negative receptor, and/or switch receptor, and/or bispecific antibody, and/or combinations thereof, the cells can be activated and expanded in number using methods known to one of skill in the art. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 and these can be used in the present disclosure as can other methods and reagents known in the art.

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20-fold to about 50-fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method includes isolating T cells and expanding the T cells. In another embodiment, the invention further includes cryopreserving the T cells prior to expansion. In yet another embodiment, the cryopreserved T cells are thawed for electroporation with the RNA encoding the chimeric membrane protein. In some embodiments, one or more of these steps is performed at modules 208 of modular biological foundry system 200 by utilizing rigid cartridge 300, docking device, gripping device, medium distribution system, or a combination thereof.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time. In some embodiments, one or more of these steps is performed at modules 208 of modular biological foundry system 200 by utilizing rigid cartridge 300, docking device, gripping device, medium distribution system, or a combination thereof.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging. Therefore, the number of population doublings of a culture is greater than the passage number. The expansion of cells (i.e., the number of population doublings) during the period between passaging depends on many factors, including but is not limited to the seeding density, substrate, medium, and time between passaging. In some embodiments, one or more of these steps is performed at modules 208 of modular biological foundry system 200 by utilizing rigid cartridge 300, docking device, gripping device, medium distribution system, or a combination thereof.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-.alpha. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, .alpha.-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37 degrees C.) and atmosphere (e.g., air plus 5% CO.sub.2).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20-fold to about 50-fold, or more. In one embodiment, human T regulatory cells are expanded via anti-CD3 antibody coated KT64.86 artificial antigen presenting cells (aAPCs). In one embodiment, human T regulatory cells are expanded via anti-CD3 antibody coated K562 artificial antigen presenting cells (aAPCs). In some embodiments, one or more of these steps is performed at modules 208 of modular biological foundry system 200 by utilizing rigid cartridge 300, docking device, gripping device, medium distribution system, or a combination thereof.

In one embodiment, the method of expanding the T cells can further includes isolating the expanded T cells for further applications. In another embodiment, the method of expanding can further include a subsequent electroporation of the expanded T cells followed by culturing. The subsequent electroporation may include introducing a nucleic acid encoding an agent, such as transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function. In some embodiments, one or more of these steps is performed at modules 208 of modular biological foundry system 200 by utilizing rigid cartridge 300, docking device, gripping device, medium distribution system, or a combination thereof.

### Example 1: Methods of Treatment of a Subject Using Cellular Engineering Targets

In some embodiments, the cellular engineering targets is modified cells (e.g., T cells). In some embodiments, a composition for immunotherapy includes the modified cells. In some embodiments, the composition includes a pharmaceutical composition and further include a pharmaceutically acceptable carrier. In some embodiments, a therapeutically effective amount of the pharmaceutical composition include the modified T cells is administered.

In one aspect, the present disclosure includes a method for adoptive cell transfer therapy including administering to a subject in need thereof a cellular engineering target including a modified T cell of the present disclosure. In another aspect, the present disclosure includes a method of treating a disease or condition in a subject including administering to a subject in need thereof a population of modified T cells

In some embodiments, a method of treating a disease or condition in a subject in need thereof includes administering to the subject a modified cell (e.g., modified T cell) of the present invention. In one embodiment, the method of treating a disease or condition in a subject in need thereof includes administering to the subject a modified cell (e.g., a modified T cell) including a subject CAR, dominant negative receptor and/or switch receptor, and/or a bispecific antibody, and/or combinations thereof. In one embodiment, the method of treating a disease or condition in a subject in need thereof includes administering to the subject a modified cell (e.g., a modified T cell) including a subject CAR (e.g., a CAR having affinity for PSMA on a target cell) and a dominant negative receptor and/or switch receptor. In one embodiment, the method of treating a disease or condition in a subject in need thereof includes administering to the subject a modified cell (e.g., a modified T cell) including a subject CAR (e.g., a CAR having affinity for PSMA on a target cell), a dominant negative receptor and/or switch receptor, and wherein the modified cell is capable of expressing and secreting a bispecific antibody.

Methods for administration of immune cells for adoptive cell therapy are known and may be used in connection with the provided methods and compositions. In some embodiments, autologous transfer conducts the cell therapy, e.g., adoptive T cell therapy, in which the cells are isolated and/or otherwise prepared from the subject who is to receive the cell therapy, or from a sample derived from such a subject. Thus, in some embodiments, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, by isolating and/or otherwise preparing the cells from a subject other than a subject who is to receive or who ultimately receives the cell therapy, e.g., a first subject. In such embodiments, the method includes administering to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the method includes treating the subject with a therapeutic agent targeting the disease or condition, e.g., the tumor, prior to administering of the cells or composition containing the cells. In some embodiments, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administering the cellular engineering target effectively treats the subject despite the subject having become resistant to another therapy.

In some embodiments, the subject is responsive to the other therapeutic agent, and treatment with the therapeutic agent reduces disease burden. In some embodiments, the subject is initially responsive to the therapeutic agent, but exhibits a relapse of the disease or condition over time. In some embodiments, the subject has not relapsed. In some such embodiments, a determination that the subject is at risk for relapse is provided, such as at a high risk of relapse, and thus the method includes administering cellular engineering target prophylactically, e.g., to reduce the likelihood of or prevent relapse. In some embodiments, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administrating the cellular engineering target effectively treats the subject despite the subject having become resistant to another therapy.

In some embodiments, the method includes administering the modified immune cells of the cellular engineering target of the present disclosure to an animal, preferably a mammal, even more preferably a human, to treat a cancer. In addition, in some embodiments, the cellular engineering target of the present invention is utilized for the treatment of any condition related to a cancer, especially a cell-mediated immune response against a tumor cell(s), where it is desirable to treat or alleviate the disease. The types of cancers with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, carcinomas amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

In some embodiments, sarcomas amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Prostate adenocarcinoma is an extremely common and lethal disease. Prostate cancer is the most common malignancy among men. Prostate cancer is the second-leading cause of cancer-related deaths among men, accounting for an estimated 10% of annual male cancer deaths. PSMA is highly expressed in malignant prostate tissue, with low-levels of expression in some normal human tissues. Under normal physiologic conditions, PSMA is expressed in the prostate gland (secretory acinar epithelium), kidney (proximal tubules), nervous system glia (astrocytes and Schwann cells), and the small intestine (jejunal brush border). PSMA is much more highly expressed in prostate epithelium and is significantly upregulated in malignant prostate tissues. PSMA expression in normal cells has been found to be 100-fold to 1000-fold less than in prostate carcinoma cells. PSMA expression increases significantly during the transformation from benign prostatic hyperplasia to prostatic adenocarcinoma. PSMA expression has been found to be directly correlated with the histologic grade of malignant prostate tissue and increases with more advanced disease (i.e. highest PSMA expression found in prostate cancer metastases in lymph node and bone).

In one embodiment, the methods of the invention are useful for treating prostate cancer, for example advanced castrate-resistant prostate cancer. It should be readily understood by one of ordinary skill in the art that any type of cancer wherein the PSMA tumor antigen is expressed, can be treated using the methods of the present invention. For example, neovascular expression of PSMA was found in non-small cell lung cancer. Accordingly, the methods of the invention may also be useful for treating non-small cell lung cancer (NSCLC).

In certain exemplary embodiments, the modified immune cells of the invention treat prostate cancer. In one embodiment, a method of the present disclosure provides a treatment for castrate-resistant prostate cancer. In one embodiment, a method of the present disclosure provides a treatment for advanced castrate-resistant prostate cancer. In one embodiment, a method of the present disclosure provides a treatment for metastatic castrate-resistant prostate cancer. In one embodiment, a method of the present disclosure provides a treatment for metastatic castrate-resistant prostate cancer, wherein the patient with metastatic castrate-resistant prostate cancer has .gtoreq.10% tumor cells expressing PSMA. In one embodiment, a method of the present disclosure provides a treatment for castrate-resistant prostate adenocarcinoma, wherein the patient has castrate levels of testosterone (e.g., <50 ng/mL) with or without the use of androgen deprivation therapy.

In certain embodiments, the method includes providing the subject with a secondary treatment. Secondary treatments include but are not limited to chemotherapy, radiation, surgery, and medications.

In some embodiments, the method includes administering the cellular engineering in dosages and routes and at times determined based on appropriate pre-clinical and clinical experimentation and trials. In some embodiments, the method includes administering cellular engineering target compositions multiple times at dosages within these ranges. The administrating of the cells of the invention includes other methods useful to treat the desired disease or condition as determined by those of skill in the art.

In some embodiments, administrating of the cellular engineering target of the present disclosure includes any convenient manner known to those of skill in the art. In some embodiments, administrating of the cellular engineering target includes aerosol inhalation, injection, ingestion, transfusion, implantation or transplantation. In some embodiments, administrating of the cellular engineering target compositions includes transarterially, subcutaneously, intradermally, intratumorally, intranodally, intramedullary, intramuscularly, by intravenous (i.v.) injection, or intraperitoneally. In some embodiments, administrating of the cellular engineering target includes injection into a site of the subject, a local disease site in the subject, a lymph node, an organ, a tumor, and the like.

In some embodiments, administrating of the cellular engineering target is at a desired dosage, which includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the method includes administering the populations or sub-types of cells, such as CD8.sup.+ and CD4.sup.+ T cells, at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, the individual populations or sub-types are present at or near a desired output ratio (such as CD4.sup.+ to CD8.sup.+ ratio), e.g., within a certain tolerated difference or error of such a ratio.

In some embodiments, the method includes administrating of the cellular engineering target at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4.sup.+ to CD8.sup.+ cells, and/or is based on a desired fixed or minimum dose of CD4.sup.+ and/or CD8.sup.+ cells.

In some embodiments, the method includes administrating of the cellular engineering target to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some embodiments about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1.times.10.sup.5 cells/kg to about 1.times.10.sup.11 cells/kg, 10.sup.4, and at or about 10.sup.11 cells/kilograms (kg) body weight, such as between 10.sup.5 and 10.sup.6 cells/kg body weight, for example, at or about 1.times.10.sup.5 cells/kg, 1.5.times.10.sup.5 cells/kg, 2.times.10.sup.5 cells/kg, or 1.times.10.sup.6 cells/kg body weight. For example, in some embodiments, the cells are administered at, or within a certain range of error of, between at or about 10.sup.4 and at or about 10.sup.9 T cells/kilograms (kg) body weight, such as between 10.sup.5 and 10.sup.6 T cells/kg body weight, for example, at or about 1.times.10.sup.5 T cells/kg, 1.5.times.10.sup.5 T cells/kg, 2.times.10.sup.5 T cells/kg, or 1.times.10.sup.6 T cells/kg body weight. In other exemplary embodiments, a suitable dosage range of modified cells for use in a method of the present disclosure includes, without limitation, from about 1.times.10.sup.5 cells/kg to about 1.times.10.sup.6 cells/kg, from about 1.times.10.sup.6 cells/kg to about 1.times.10.sup.7 cells/kg, from about 1.times.10.sup.7 cells/kg about 1.times.10.sup.8 cells/kg, from about 1.times.10.sup.8 cells/kg about 1.times.10.sup.9 cells/kg, from about 1.times.10.sup.9 cells/kg about 1.times.10.sup.10 cells/kg, from about 1.times.10.sup.10 cells/kg about 1.times.10.sup.11 cells/kg. In an exemplary embodiment, a suitable dosage for use in a method of the present disclosure is about 1.times.10.sup.8 cells/kg. In an exemplary embodiment, a suitable dosage for use in a method of the present disclosure is about 1.times.10.sup.7 cells/kg. In other embodiments, a suitable dosage is from about 1.times.10.sup.7 total cells to about 5.times.10.sup.7 total cells. In some embodiments, a suitable dosage is from about 1.times.10.sup.8 total cells to about 5.times.10.sup.8 total cells. In some embodiments, a suitable dosage is from about 1.4.times.10.sup.7 total cells to about 1.1.times.10.sup.9 total cells. In an exemplary embodiment, a suitable dosage for use in a method of the present disclosure is about 7.times.10.sup.9 total cells. In an exemplary embodiment, a suitable dosage is from about 1.times.10.sup.7 total cells to about 3.times.10.sup.7 total cells.

In some embodiments, the dose of total cells and/or dose of individual sub-populations of cells is within a range of between at or about 1.times.10.sup.5 cells/m.sup.2 to about 1.times.10.sup.11 cells/m.sup.2. In an exemplary embodiment, the dose of total cells and/or dose of individual sub-populations of cells is within a range of between at or about 1.times.10.sup.7/m.sup.2 to at or about 3.times.10.sup.7/m.sup.2. In an exemplary embodiment, the dose of total cells and/or dose of individual sub-populations of cells is within a range of between at or about 1.times.10.sup.8/m.sup.2 to at or about 3.times.10.sup.8/m.sup.2. In some embodiments, the dose of total cells and/or dose of individual sub-populations of cells is the maximum tolerated dose by a given patient.

In some embodiments, the method includes administrating the cellular engineering target at or within a certain range of error of between at or about 10.sup.4 and at or about 10.sup.9 CD4.sup.+ and/or CD8.sup.+ cells/kilograms (kg) body weight, such as between 10.sup.5 and 10.sup.6 CD4.sup.+ and/or CD8.sup.+ cells/kg body weight, for example, at or about 1.times.10.sup.5 CD4.sup.+ and/or CD8.sup.+ cells/kg, 1.5.times.10.sup.5 CD4.sup.+ and/or CD8.sup.+ cells/kg, 2.times.10.sup.5 CD4.sup.+ and/or CD8.sup.+ cells/kg, or 1.times.10.sup.6 CD4.sup.+ and/or CD8.sup.+ cells/kg body weight. In some embodiments, the cells are administered at or within a certain range of error of, greater than, and/or at least about 1.times.10.sup.6, about 2.5.times.10.sup.6, about 5.times.10.sup.6, about 7.5.times.10.sup.6, or about 9.times.10.sup.6 CD4.sup.+ cells, and/or at least about 1.times.10.sup.6, about 2.5.times.10.sup.6, about 5.times.10.sup.6, about 7.5.times.10.sup.6, or about 9.times.10.sup.6 CD8+ cells, and/or at least about 1.times.10.sup.6, about 2.5.times.10.sup.6, about 5.times.10.sup.6, about 7.5.times.10.sup.6, or about 9.times.10.sup.6 T cells. In some embodiments, the cells are administered at or within a certain range of error of between about 10.sup.8 and 10.sup.12 or between about 10.sup.10 and 10.sup.11 T cells, between about 10.sup.8 and 10.sup.12 or between about 10.sup.10 and 10.sup.11 CD4.sup.+ cells, and/or between about 10.sup.8 and 10.sup.12 or between about 10.sup.10 and 10.sup.11 CD8.sup.+ cells.

In some embodiments, the method includes administrating the cellular engineering target with a toleration range of a desired output ratio of multiple cell populations or sub-types, such as CD4+ and CD8+ cells or sub-types. In some embodiments, the desired ratio is a specific ratio or can be a range of ratios, for example, in some embodiments, the desired ratio (e.g., ratio of CD4.sup.+ to CD8.sup.+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some embodiments, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the method includes administrating the cellular engineering target a dose of modified cells in a single dose or multiple doses. In some embodiments, administrating the cellular engineering includes multiple doses, e.g., once a week or every 7 days, once every 2 weeks or every 14 days, once every 3 weeks or every 21 days, once every 4 weeks or every 28 days. In an exemplary embodiment, administrating the cellular engineering includes a single dose of modified cells, such as by rapid intravenous infusion.

In some embodiments, for the prevention or treatment of disease, the appropriate dosage depends on the type of disease, the type of cells or recombinant receptors, the severity and course of the disease, whether administrating the cells for preventive or therapeutic purposes, previous therapy, the subject's clinical history and response to the cellular engineering target, and the discretion of the attending physician. In some embodiments, the method includes administrating the compositions and cells once or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods include administration of a chemotherapeutic agent.

In some embodiments, the method includes determining the biological activity of the cellular engineering target, e.g., by any of a number of known methods. In some embodiments, one or more parameters utilized in such a determination include specific binding of an engineered or natural T cell or other immune cell to antigen, in vivo, e.g., by imaging, or ex vivo, e.g., by ELISA or flow cytometry. In certain embodiments, the method includes determining the ability of the engineered cells to destroy target cells using any suitable method known in the art, such as cytotoxicity assays. In certain embodiments, the method includes determining the biological activity of the cells by assaying expression and/or secretion of one or more cytokines, such as CD 107a, IFNy, IL-2, and TNF. In some embodiments, the method includes determining the biological activity by assessing clinical outcome, such as reduction in tumor burden or load.

In some embodiments, the method includes providing a specific dosage regimen that includes a lymphodepletion step prior to the administration of the modified T cells. In an exemplary embodiment, the lymphodepletion step includes administrating cyclophosphamide and/or fludarabine.

In some embodiments, the administrating of lymphodepletion includes administrating cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day). In an exemplary embodiment, the dose of cyclophosphamide is about 300 mg/m.sup.2/day. In some embodiments, the lymphodepletion step includes administration of fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, the dose of fludarabine is about 30 mg/m.sup.2/day.

In some embodiment, the administrating of lymphodepletion includes administrating cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day), and fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, the administrating of lymphodepletion includes administrating cyclophosphamide at a dose of about 300 mg/m.sup.2/day, and fludarabine at a dose of about 30 mg/m.sup.2/day.

In an exemplary embodiment, a subject has a diagnosis for castrate-resistant prostate cancer, the method includes administrating lymphodepleting chemotherapy prior to administrating of the modified T cellular engineering target. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion about 3 days (.+−.1 day) prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion up to 4 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 4 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 3 days prior to administration of the modified T cells. In an exemplary embodiment, for a subject having castrate-resistant prostate cancer, the subject receives lymphodepleting chemotherapy including at or about 500 mg/m.sup.2 to at or about 1 g/m.sup.2 of cyclophosphamide by intravenous infusion 2 days prior to administration of the modified T cells.

In an exemplary embodiment, the method includes, a subject having castrate-resistant prostate cancer, administrating lymphodepleting chemotherapy including 300 mg/m.sup.2 of cyclophosphamide by intravenous infusion 3 days prior to administrating the modified T cells. In an exemplary embodiment, for a subject having castrate-resistant prostate cancer, the method includes administrating lymphodepleting chemotherapy including 300 mg/m.sup.2 of cyclophosphamide by intravenous infusion for 3 days prior to administrating the modified T cells.

In an exemplary embodiment, for a subject having castrate-resistant prostate cancer, the method includes administrating lymphodepleting chemotherapy including fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, for a subject having castrate-resistant prostate cancer, the method includes administrating lymphodepleting chemotherapy including fludarabine at a dose of 30 mg/m.sup.2 for 3 days.

In an exemplary embodiment, for a subject having castrate-resistant prostate cancer, the method includes administrating lymphodepleting chemotherapy including cyclophosphamide at a dose of between about 200 mg/m.sup.2/day and about 2000 mg/m.sup.2/day (e.g., 200 mg/m.sup.2/day, 300 mg/m.sup.2/day, or 500 mg/m.sup.2/day), and fludarabine at a dose of between about 20 mg/m.sup.2/day and about 900 mg/m.sup.2/day (e.g., 20 mg/m.sup.2/day, 25 mg/m.sup.2/day, 30 mg/m.sup.2/day, or 60 mg/m.sup.2/day). In an exemplary embodiment, for a subject having castrate-resistant prostate cancer, the method includes administrating lymphodepleting chemotherapy including cyclophosphamide at a dose of about 300 mg/m.sup.2/day, and fludarabine at a dose of 30 mg/m.sup.2 for 3 days.

It is known in the art that one of the adverse effects following infusion of CAR T cells is the onset of immune activation, known as cytokine release syndrome (CRS). CRS is immune activation resulting in elevated inflammatory cytokines. Clinical and laboratory measures range from mild CRS (constitutional symptoms and/or grade-2 organ toxicity) to severe CRS (sCRS; grade.gtoreq.3 organ toxicity, aggressive clinical intervention, and/or potentially life threatening). Clinical features include high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the present disclosure provides for, following the diagnosis of CRS, appropriate CRS management strategies that mitigate one or more physiological symptoms of uncontrolled inflammation without dampening the antitumor efficacy of the cellular engineering target (e.g., CAR T cells). CRS management strategies are known in the art. For example, in some embodiments, the method includes administrating systemic corticosteroids to rapidly reverse symptoms of sCRS (e.g., grade 3 CRS) without compromising initial antitumor response.

In some embodiments, the method includes administrating an anti-IL-6R antibody. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administrating of tocilizumab has demonstrated near-immediate reversal of CRS.

In some embodiments, the method includes selecting and treating a subject having failed at least one prior course of standard of cancer therapy. For example, a suitable subject may have had a confirmed diagnosis of relapsed prostate cancer. In some embodiments, the method includes selecting and treating a subject having had at least one prior course of standard of cancer therapy. For example, a suitable subject may have had prior therapy with at least one standard 17.alpha. lyase inhibitor or second-generation anti-androgen therapy for the treatment of metastatic castrate resistant prostate cancer.

In an exemplary embodiment, a suitable subject is a subject having metastatic castrate resistant prostate cancer. In an exemplary embodiment, a suitable subject is a subject having metastatic castrate resistant prostate cancer having .gtoreq.10% tumor cells expressing PSMA as determined by immunohistochemistry analysis on fresh tissue.

In some embodiments, a suitable subject is a subject that has radiographic evidence of osseous metastatic disease and/or quantifiable, non-osseous metastatic disease (nodal or visceral).

In some embodiments, a suitable subject includes an ECOG performance status of 0-1.

In some embodiments, a suitable subject exhibits adequate organ function, as defined by: serum creatinine.ltoreq.1.5 mg/dl or creatinine clearance.gtoreq.60 cc/min; and/or serum total bilirubin<1.5.times.ULN; serum ALT/AST<2.times.ULN.

In some embodiments, a suitable subject exhibits adequate hematologic reserve as defined by: Hgb>10 g/dl; PLT>100 k/ul; and/or ANC>1.5 k/ul.

In some embodiments, a suitable subject is not transfusion dependent.

In some embodiments, a suitable subject is a subject that has evidence of progressive castrate resistant prostate adenocarcinoma, as defined by: castrate levels of testosterone (<50 ng/ml) with or without the use of androgen deprivation therapy; and/or evidence of one of the following measures of progressive disease: soft tissue progression by RECIST 1.1 criteria, osseous disease progression with 2 or more new lesions on bone scan (as per PCWG2 criteria), increase in serum PSA of at least 25% and an absolute increase of 2 ng/ml or more from nadir (as per PCWG2 criteria).

In some embodiments, a suitable subject has had previous treatment with at least one second-generation androgen signaling inhibitor. In some embodiments, a suitable subject has had previous treatment with abiraterone. In some embodiments, a suitable subject has had previous treatment with enzalutamide.

In some embodiments, a suitable subject includes .gtoreq.10% tumor cells expressing PSMA by immunohistochemistry (IHC) on a metastatic tissue biopsy.

In some embodiments, a suitable subject includes radiographic evidence for metastatic disease (osseous or nodal/visceral).

In some embodiments, a suitable subject includes .ltoreq.4 lines of therapy for metastatic CRPC.

Additional details and information regarding the manufacture of cellular engineering targets can be found at U.S. Pat. No. 10,780,120, entitled “Prostate-specific membrane antigen cars and methods of use thereof,” filed Mar. 5, 2019; U.S. Pat. No. 10,839,945, entitled “Cell processing method,” filed Jul. 6, 2015; U.S. Pat. No. 10,428,351, entitled “Methods for transduction and cell processing,” filed Nov. 4, 2015; U.S. Pat. No. 10,877,055, entitled “Parallel cell processing method and facility,” filed Jan. 11, 2019, each of which is hereby incorporated by reference in its entirety for all purposes.

FIG. 2 depicts a block diagram of a distributed computer system (e.g., computer system 100) according to some embodiments of the present disclosure. The computer system 100 at least facilitates communicating one or more instructions for fabricating an engineered cell, such as a cell therapy.

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs) 274, a network or other communications interface 284, and memory 192.

In some embodiments, the computer system 100 includes a user interface 278. The user interface 278 typically includes a display 282 for presenting media. In some embodiments, the display 282 is integrated within the computer systems (e.g., housed in the same chassis as the CPU 274 and memory 192). In some embodiments, the computer system 100 includes one or more input device(s) 280, which allow a subject to interact with the computer system 100. In some embodiments, input devices 280 include a keyboard, a mouse, and/or other input mechanisms. Alternatively, or in addition, in some embodiments, the display 282 includes a touch-sensitive surface (e.g., where display 282 is a touch-sensitive display or computer system 100 includes a touch pad).

In some embodiments, the computer system 100 presents media to a user through the display 282. Examples of media presented by the display 282 include one or more images, a video, audio (e.g., waveforms of an audio sample), or a combination thereof. In typical embodiments, the one or more images, the video, the audio, or the combination thereof is presented by the display 282 through a client application. In some embodiments, the audio is presented through an external device (e.g., speakers, headphones, input/output (I/O) subsystem, etc.) that receives audio information from the computer system 100 and presents audio data based on this audio information. In some embodiments, the user interface 278 also includes an audio output device, such as speakers or an audio output for connecting with speakers, earphones, or headphones.

Memory 192 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 192 may optionally include one or more storage devices remotely located from the CPU(s) 274. Memory 192, or alternatively the non-volatile memory device(s) within memory 192, includes a non-transitory computer readable storage medium. Access to memory 192 by other components of the computer system 100, such as the CPU(s) 274, is, optionally, controlled by a controller. In some embodiments, memory 192 can include mass storage that is remotely located with respect to the CPU(s) 274. In other words, some data stored in memory 192 may in fact be hosted on devices that are external to the computer system 100, but that can be electronically accessed by the computer system 100 over an Internet, intranet, or other form of network 106 or electronic cable using communication interface 284.

In some embodiments, the memory 192 of the computer system 100 stores:
an operating system 1920 (e.g., ANDROID, iOS, DARWIN, RTXC, LINUX, UNIX, OS X, WINDOWS, or an embedded operating system such as VxWorks) that includes procedures for handling various basic system services;
an electronic address 104 associated with the computer system 100 that identifies the computer system 100 (e.g., within the communication network 106);
an instrument library 106 storing a record of a plurality of instruments 300 found at a modular clean room biological foundry system; and
a module library for storing a record of a plurality of instruments 300 found at a modular clean room biological foundry system.

As indicated above, an electronic address 104 is associated with the computer system 100. The electronic address 104 is utilized to at least uniquely identify the computer system 100 from other devices and components of the distributed system 10, such as other devices having access to the communications network 106.

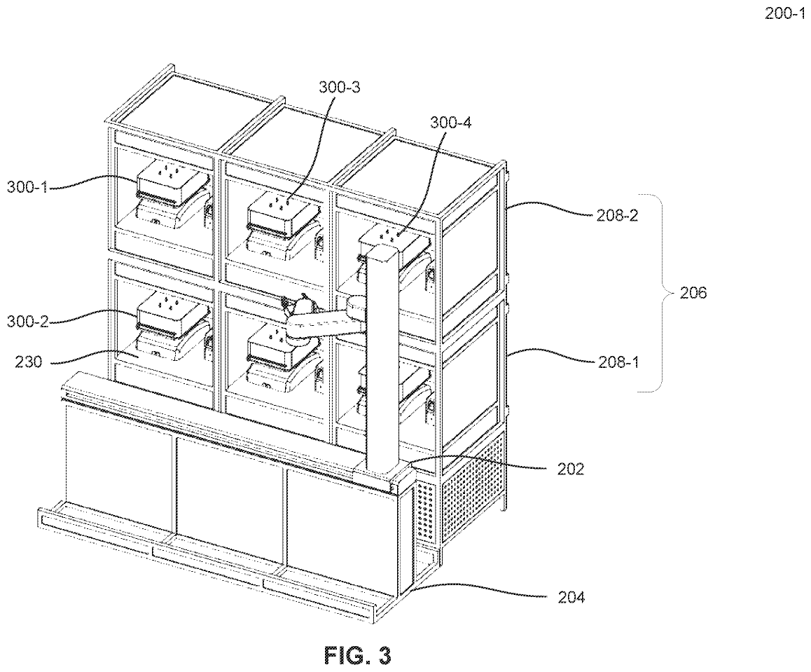

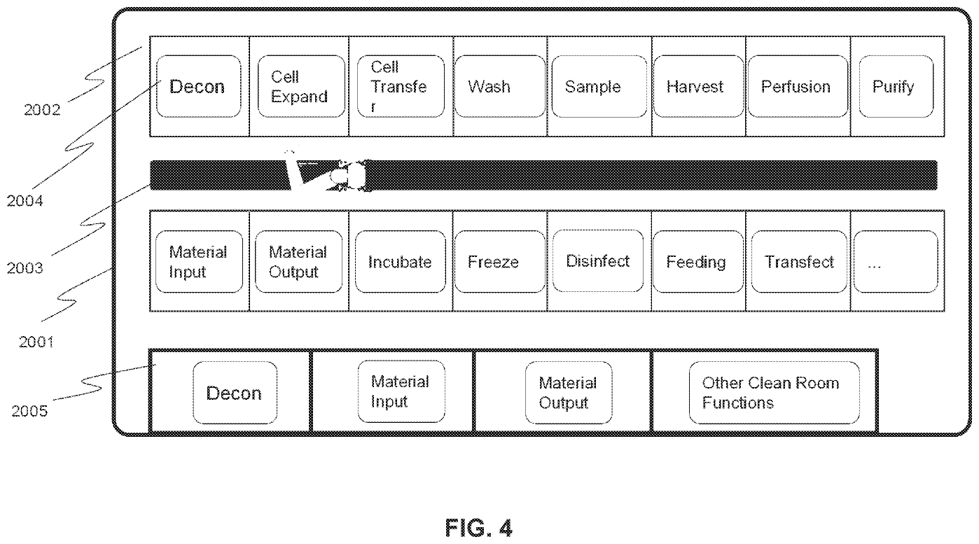

Referring to FIGS. 3 and 4, an exemplary modular clean room biological foundry system 2002 is provided.

Accordingly, one aspect of the present disclosure is directed to providing systems, methods, and apparatuses that facilitates providing a modular biological foundry system. The modular biological foundry system includes a controller and a communications interface that is in electrical communication with the controller. Moreover, the modular biological foundry system includes a plurality of peripheral devices, which in turn includes an articulated handling robot and a power supply. The articulated handling robot is configured to move a cell therapy cartridge between at least a first biological foundry instrument and a second biological foundry instrument in a plurality of biological foundry instruments configured to produce a portion of the one or more cellular engineering targets. The modular system includes a frame surrounding the articulated handling robot. The frame includes at least two modules in a plurality of modules. A first module in the at least two modules is configured to accommodate a respective biological foundry instrument in the plurality of biological foundry instructions. Moreover, each module in the plurality of modules includes a plurality of elongated members. Each module in the plurality of modules further includes a first plurality of coupling mechanisms for coupling at least two elongated members in the plurality of elongated members. Additionally, each module in the plurality of modules includes a second plurality of coupling mechanisms for removably coupling a respective elongated member in the plurality of elongated members with the frame. Furthermore, each module in the plurality of modules includes a plurality of walls engaged with and supported by the at least two elongated members in the plurality of elongated members. Accordingly, the plurality of walls forms an internal volume of a respective module sealed from an environment, such that a modular biological foundry system is provided.

In some embodiments, the modular biological foundry system further includes a transport path coupled to the articulated handling robot and in electrical communication with the communications interface. The transport path extends from a first end portion of a first wall in the plurality of walls to a second end portion of a second wall in the plurality of walls.

Turning to FIG. 3 through FIG. 6 with the foregoing in mind, consider that a manufacturing process for a cellular engineering target is divided into a plurality of steps. In each step, a different manufacturing task or sub-process is carried out. For instance, in some embodiments, the different manufacturing task includes incubation of cell culture, isolation of one or more types of cells, viral transduction, freezing, thawing, etc. Accordingly, the present disclosure provides a modular clean room biological foundry system that utilizes a separate module (e.g., module 208-1 of FIG. 3) to conduct each step of the manufacturing process.

In some embodiments, the modular clean room biological foundry system is configured to provide a space where necessary functions of different rooms or areas in a Good Manufacturing Practice (GMP) facility 2001 are performed by an automated material handling system 2003 (e.g., articulated robot), another automated system such as (but not limited to) a gantry, a dial, a conveyor belt, a machine chassis, or a combination thereof. However, the present disclosure is not limited thereto. In some embodiments, the modular clean room biological foundry system include one or more floors. In some embodiments, the miniature rooms 2004, also called “modules” herein, are arranged rectangularly (e.g., as shown in FIG. 4). In some embodiments, the rooms are arranged in any geometries such as a ring, a grid, or a star shape.

In some embodiments, by having GMP-necessary functions of different rooms 2005 miniaturized into modules 2004 and performed by a modular clean room biological foundry system 2003. In some embodiments, the airspace is separated. In some embodiments, cleaner, clean room classification is achieved inside the modular clean room biological foundry system. In some embodiments, the separate modules within the modular clean room biological foundry system enable co-location in one overall room of different processes or products. In some embodiments, the separate modules do not co-locate within one room due to GMP concerns such as (but not limited to) cross contamination, contamination, and accidental mix-up. However, the present disclosure is not limited thereto.

In some embodiments, the modules include decontamination, material input, material output, cell expansion, cell transfer, incubation, freezing, sampling, disinfecting, transfection, purification, feeding, harvesting, perfusion, washing, or a combination thereof.

In some embodiments, the decontamination module is utilized to ensure no contamination from the outside is brought inside the module. In some embodiments, by concentrating the entry point for materials entering the modular clean room biological foundry system, the risk of contamination is reduced, and the modular clean room biological foundry system is monitored and controlled. In some embodiments, the decontamination in the module employs autoclave, ultraviolet light, irradiation, and chemical sanitizer, vapor of alcohol, or hydrogen peroxide or other agents, disinfectant, sterilant, or a combination thereof. In some embodiments, sets of interlocking doors are used to control airflow, differential pressure, air changes, or a combination thereof controlled to maintain clean room classification in the modular clean room biological foundry system.

Referring to FIG. 5, an embodiment where a modular clean room biological foundry system 2001 that includes a plurality of modular clean room biological foundry systems 2002 arranged in an exemplary configuration having multiple floors is provided. In some embodiments, each modular clean room biological foundry system includes all of the necessary GMP functions. However, the present disclosure is not limited thereto. In some embodiments, each modular clean room biological foundry system is considered a self-contained clean room.

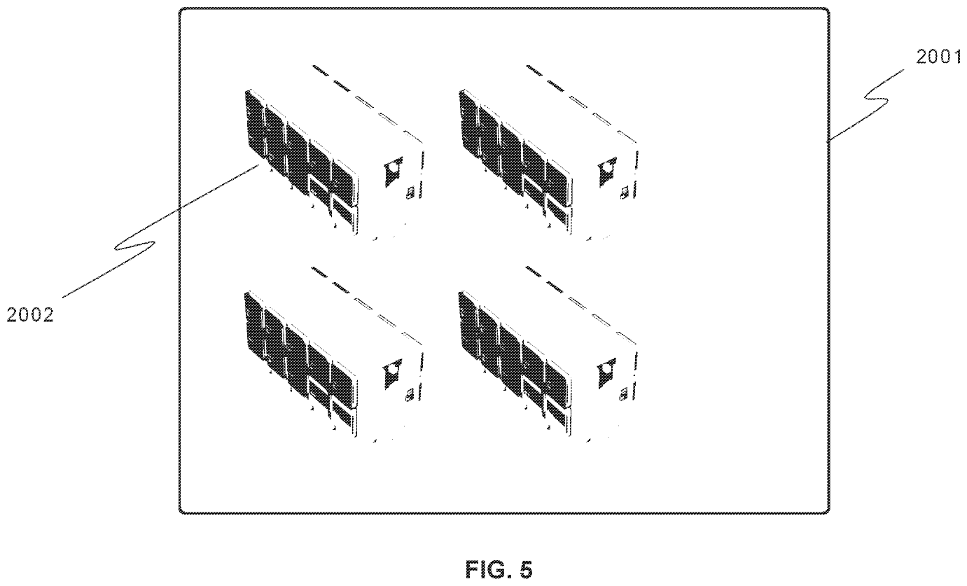

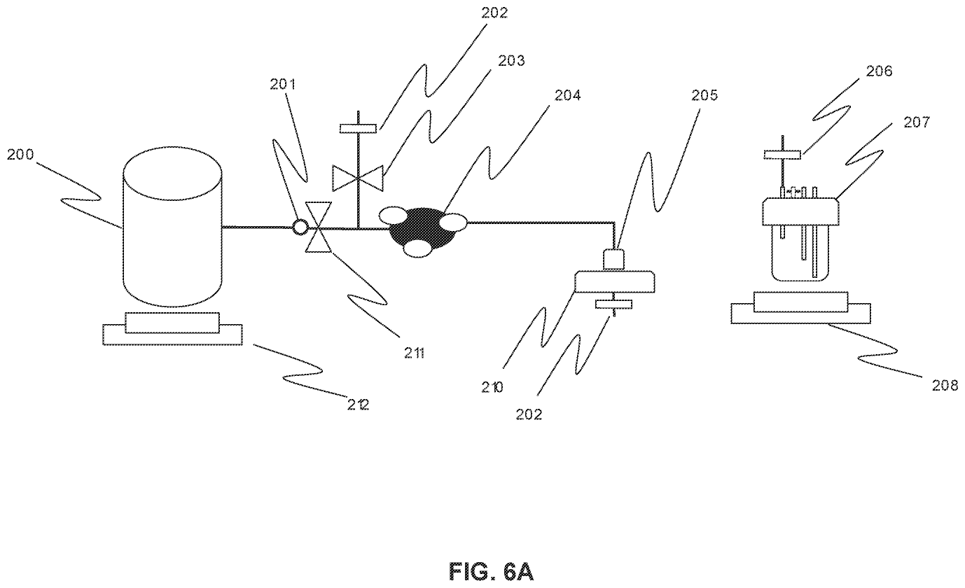

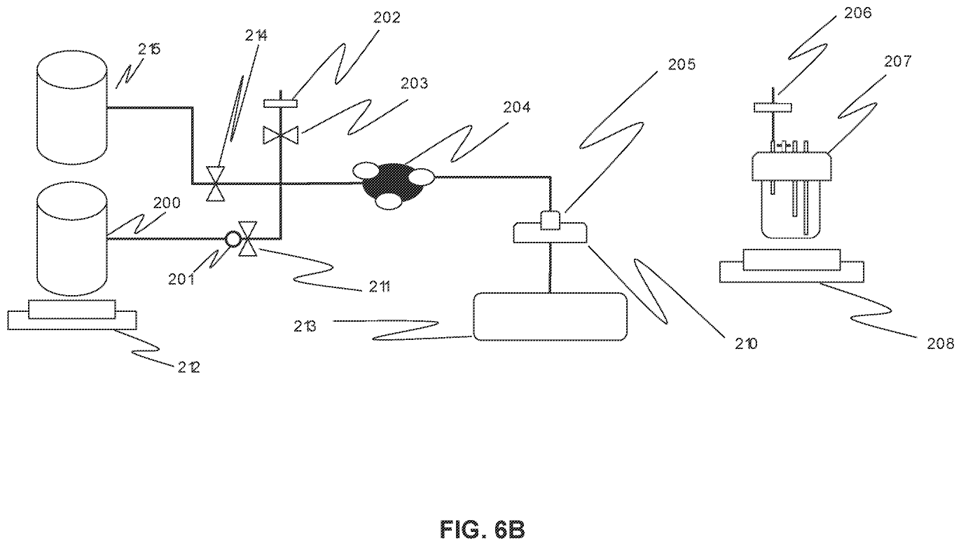

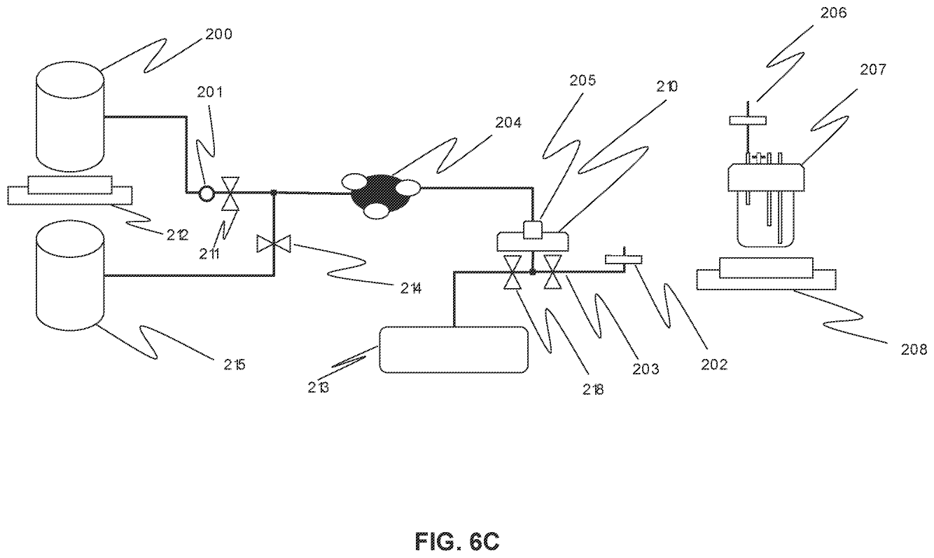

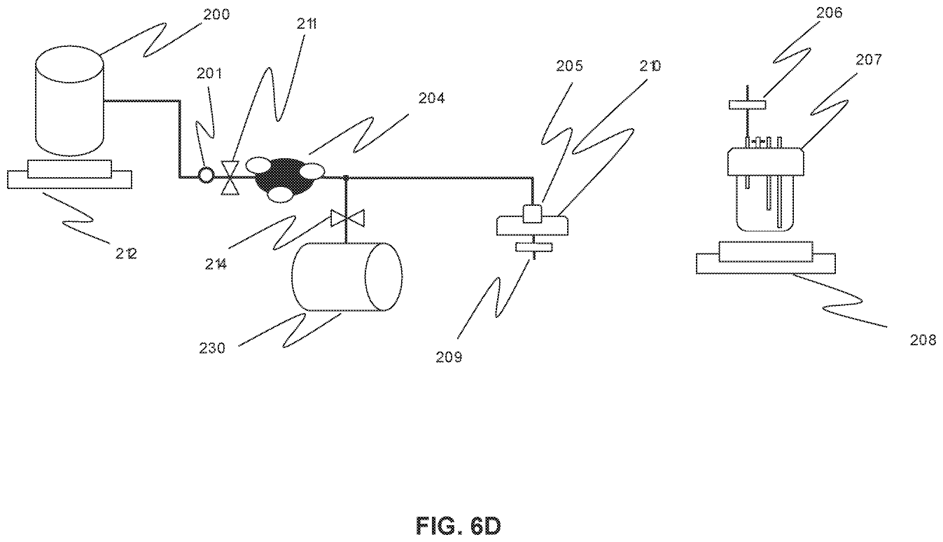

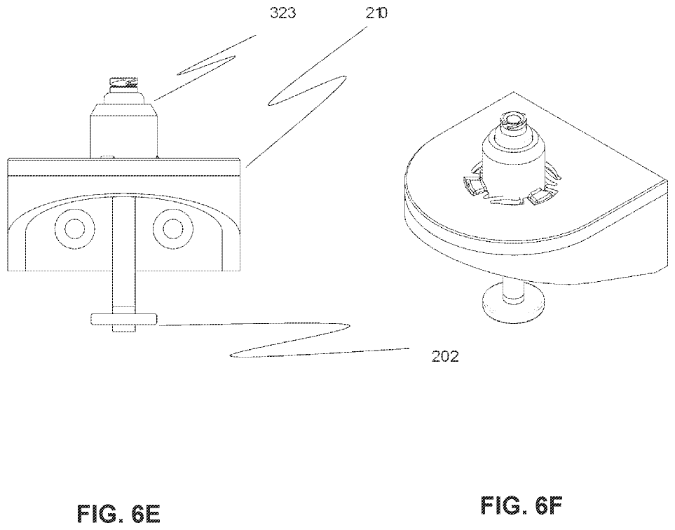

Referring briefly to FIGS. 6A through 6F, in some embodiments, the present disclosure provides a medium distribution system including a manifold, an apparatus, and/or a method for aseptic control of one or more mediums (e.g., one or more reservoirs) from one or more (e.g., reservoirs 200 of FIG. 6A, into a plurality of rigid cartridges (e.g., two or more rigid cartridges that accommodate a soft body bioreactor) 207. In some embodiments, the medium distribution system performs a method to manufacture a cell therapy using a rigid cartridge, a docking device, a gripping device, or a combination thereof without contamination of multi-use tubing, such as by using an air buffer delivery system of a manifold. In FIG. 6A, this medium distribution system is depicted in a first (e.g., initial) configuration with one or more mediums (e.g., reagents) stored in reservoirs 200. In some embodiments, each reservoir includes an interior volume that is a sufficiently large in volume to supply a respective medium to a plurality of rigid cartridges and/or in an environmentally controlled environment. In some embodiments, from this reservoir, a flow of medium is controlled by peristaltic pump 204 via non-fluid contacting valving mechanism 211 such as a pinch valve. Additional details and information regarding the peristaltic pump 204 and non-fluid contacting valving mechanism is found at U.S. patent application Ser. No. 18/154,950, entitled “System, Method, and Apparatus for Manufacture of Engineered Cells,” filed Jan. 16, 2023, which is hereby incorporated by reference in its entirety for all purposes. In some embodiments, upstream from the non-fluid contacting valving mechanism (e.g., set up in line just before the non-fluid contacting valving mechanism) is a sensor, such as optical and/or ultrasonic fluid sensor 201, which is utilized to, at least, ensure a threshold mass and/or volume of the medium is dispensed through an outlet port of the manifold. In some embodiments, a secondary non-fluid contacting valving mechanism to an environment (e.g., external air) 203 is closed and in-line with air filter 202 to prevent outside contaminants from entering the system. However, the present disclosure is not limited thereto. As used herein, the non-fluid contacting valving mechanism in line with the sensor and medium container is also referred to as “the fluid valve.” Moreover, as used herein, the non-fluid contacting valving mechanism in line with the outside air and filter is also referred to as “the air valve.” In some embodiments, these air valve and the fluid valve are combined to a single high-performance liquid chromatography (HPLC) fluid valve. In some embodiments, the HPLC fluid valve is disposed at a junction of the fluid valve and the air valve, which allows the HPLC fluid valve to perform a function of the fluid valve and the air valve. In some embodiments, an output of the manifold has a rigid connector (e.g., a needle free connector) and is docked onto a docking device 210. Referring briefly to FIGS. 6E and 6F, in some such embodiments, this docking device is utilized for establishing a sterile path within this system. In some embodiments, this docking device includes rigid connector (e.g., a needly free connector) 323 in line with filter 202 (e.g., air filter). In some embodiments, the manifold includes a sensor, such as one or more load cells or similar fluid volume systems 212, is integrated into a control system of the computer system, such as in order to determine if a volume changes in a first rigid cartridge and/or a reservoir. However, the present disclosure is not limited thereto.

In some embodiments, an initiation of transfer of the medium through the manifold begins when the tube docker 205 is disengaged from the tube dock 210 and connected to the needle free connector 323 on an upper end portion of the bioreactor 207 (e.g., as operated by a robotic system with appropriate gripper accessory). Additional details and information regarding gripper accessory of the present disclosure can be found at U.S. Pat. No. 10,773,392, entitled “Flexure Gripping Device,” filed Mar. 7, 2019; U.S. patent application Ser. No. 18/154,950, entitled “System, Method, and Apparatus for Manufacture of Engineered Cells,” filed Jan. 16, 2023, each of which is hereby incorporated by reference in its entirety. In some embodiments, connectors 323 and 331 inhibit an ingress of air into the apparatus throughout an epoch during which the connector is in transit (e.g., once connection to the rigid cartridge, the sterile path is reinstated). In some such embodiments, the fluid valve 211 is configured (e.g., in accordance with one or more instructions by controller of computer system 100) to open and the pump 204 begins to provide the medium along a flow path from the reservoir 200 to the rigid cartridge 207. In some embodiments, the medium is dispensed in an amount in accordance with a determination, such as by the controller, that a threshold volume and/or a threshold mass of the medium disposed by the manifold is satisfied. In some embodiments, the amount is provided by one or more sensors, such as from the flow or sensor 201 (e.g., bubble sensor) and the load cell 212. In some embodiments, the pump 204 is controlled (e.g., by controller of computer system 100) to dispense a volume of a first medium to be disposed into the rigid cartridge along the fluid path and past the sensor 201 and fluid valve 211. In some embodiments, after this threshold condition of the volume is satisfied, the flow path is changed by closing fluid valve 211 and opening a flow path to sterile air through air valve 203. In some embodiments, the pump resumes providing the medium towards the rigid cartridge, delivering a bolus of fluid (e.g., as pushed by sterile air behind the bolus of fluid). In this way in some such embodiments, the correct volume of fluid is delivered without creating a direct fluid path between the clean medium, which is used for a different patient and the rigid cartridge with a patient-specific cells. In some such embodiments, the medium is injected above a water line of the rigid cartridge and through a particulate filter 206, which is configured with a first size that is less than a second size, which restrict cellular matter of at least the second size from traveling through the particulate filter. In some embodiments, after this fluid is dispensed, the tube, filled with sterile air, is disconnected and attached back to the tubing dock. Accordingly, in some embodiments, the line from the fluid valve 211 to the medium volume 200 is purged of remaining medium either via pumping backwards with the medium valve open or by opening both air and medium valves 203 and 211 and allowing regent to gravity drain into the reservoir 200. In some such embodiments, the pump 204 only rotates in one direction, which is a risk mitigation in designing a single-fault tolerant safe system.

Referring briefly to FIG. 6B, in some embodiments, the present disclosure provides a medium distribution system including a manifold, an apparatus, and/or a method for aseptic control of one or more mediums (e.g., one or more reservoirs) from one or more (e.g., reservoirs 200 of FIG. 6A, into a plurality of rigid cartridges (e.g., two or more rigid cartridges that accommodate a soft body bioreactor) 207. In some embodiments, the medium distribution system allows for providing the one or more mediums without contaminating the multi-use tubing or regent reservoir using an air buffer delivery and a buffer 215 flush of the multi-use tubing into a waste reservoir 213. In some embodiments, the medium distribution system of FIG. 6B utilizes similar components and methods as the medium distribution system of FIG. 6A, expect with one or more differences as described below. In some embodiments, the medium distribution system of FIG. 6B includes an additional inlet medium branch that starts with a buffer or line cleaning flush solution reservoir 215, which has an inline valve 214, or a valving junction control like an HPLC valve. In some embodiments, a lower end portion of the tube docker 210 is connected inline to a waste reservoir 213.

In some embodiments, the medium distribution system FIG. 6B utilizes similar components and methods as the medium distribution system of FIG. 6A, expect the inclusion of a final series of steps in which, after regent is returned to the reservoir and valve 211 is closed, valve 214 is open and buffer is pumped via pump 204 into the system and collected into the waste reservoir 213. In some embodiments, a series of backflows, one or more filters, one or more one-way valves, one or more contaminant reduction mechanisms, or a combination thereof is utilized within the system to mitigate waste contamination concerns. In some embodiments, after pumping the buffer through the line, valve 214 closes and valve 203 opens, and the pump pushes the rest of the cleaning fluid along the tubing path and into the waste reservoir 213.

Referring briefly to FIG. 6C, in some embodiments, the present disclosure provides a medium distribution system including a manifold, an apparatus, and/or a method for aseptic control of one or more mediums (e.g., one or more reservoirs) from one or more (e.g., reservoirs 200 of FIG. 6A, into a plurality of rigid cartridges (e.g., two or more rigid cartridges that accommodate a soft body bioreactor) 207. In some embodiments, the medium distribution system provides the one or more mediums without cross contamination with a buffer or line cleaning flush solution 215.

In some embodiments, components for this medium distribution system of FIG. 6C differs from the medium distribution system FIG. 6A and/or FIG. 6B, by the exchange of the airline (valving mechanism 203, 218 and air filter 202) from the front of the pump 204 to within the docker system. In some embodiments, implications of such changes mean instead of the delivery of fluid as a bolus surrounded by air, media is dispensed more closely with feedback from the sensor (e.g., load cell 208). For instance, in some such embodiments, the entire fluid bolus back to the valve is then considered contaminated, and when the tube holder docks onto the tube docker that entire column of fluid is washed via pump moving buffer fluid into the waste reservoir, out of the tube. In some embodiments, air is then pumped back into the system through filter 202 up to the valves 211 before the priming of the next fluid delivery.

Referring briefly to FIG. 6D, in some embodiments, the present disclosure provides a medium distribution system including a manifold, an apparatus, and/or a method for aseptic control of one or more mediums (e.g., one or more reservoirs) from one or more (e.g., reservoirs 200 of FIG. 6A, into a plurality of rigid cartridges (e.g., two or more rigid cartridges that accommodate a soft body bioreactor) 207. In some embodiments, the medium distribution system provides the one or more mediums without cross contamination with a non-fluid contacting pump 204 and compressed air delivery system 230 with actuating valve 214.

In some embodiments, this system of FIG. 6D utilizes similar components and methods as the medium distribution system of FIG. 6A, but instead of having the air pumped into the system through a line, the force from the compressed air in conjunction with the pump motion would establish that same fluid motion.

In various embodiments, the present disclosure is directed to providing an apparatus that includes a cartridge for manufacture of a cell therapy. Referring to FIGS. 7A through 15, a variety of exemplary apparatuses (e.g., instrument 300-1 of FIG. 2) including a rigid cartridge, a docking device, a gripping device, a manifold, or a combination thereof are provided, which facilitates sampling of a cellular engineering targeted accommodated by a rigid cartridge and/or a soft body bioreactor, which is performed automatically by articulated handing robot 204 within the modular biological foundry system. Accordingly, the apparatus of the present disclosure provides repeatability of automatic systems that ensures consistency of manufacture of a cellular engineering target, such that multiple samples can be effectively compared, and the variations reflect variations in the cellular engineering target. As such, the apparatus of the present disclosure makes reduces complexity and increases safety to ensure and control the quality of the cellular engineering target.

In some embodiments, rigid cartridge 207 is utilized to store a material utilized in the manufacture of a cellular engineering target, such as cell culture media, reagents, pharmaceutical ingredients, etc.). In some embodiments, the rigid cartridge is initially an empty container that will then accommodate a material, such as waste material produced as an instrument accommodated by the module performs a manufacturing task. Accordingly, a size of the rigid cartridges allows a user to insert new rigid cartridges and remove old rigid cartridges from the modular biological foundry system without aid of a robot and/or without affecting the sterility or the clean-room conditions inside the modular biological foundry system. However, the present disclosure is not limited thereto.

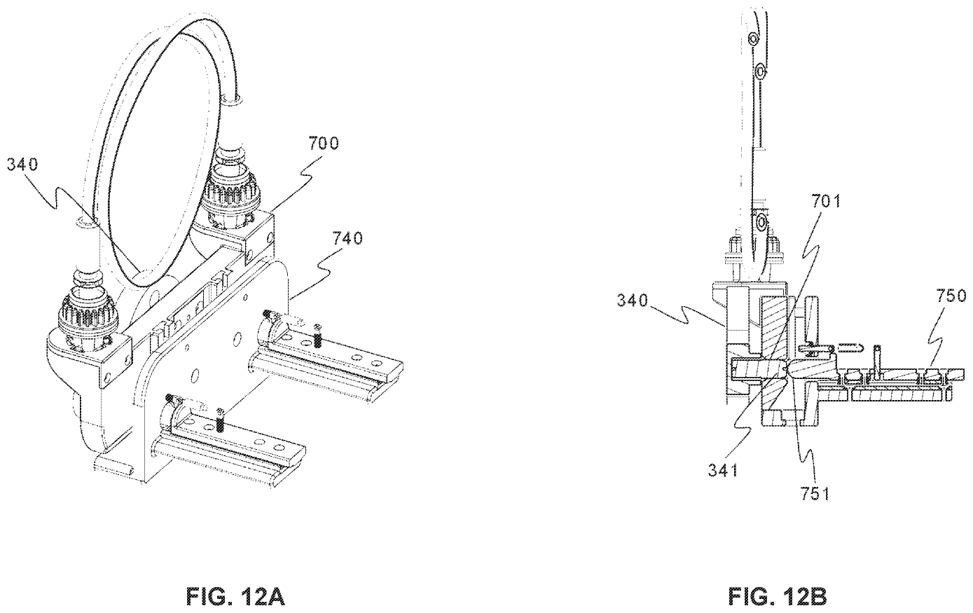

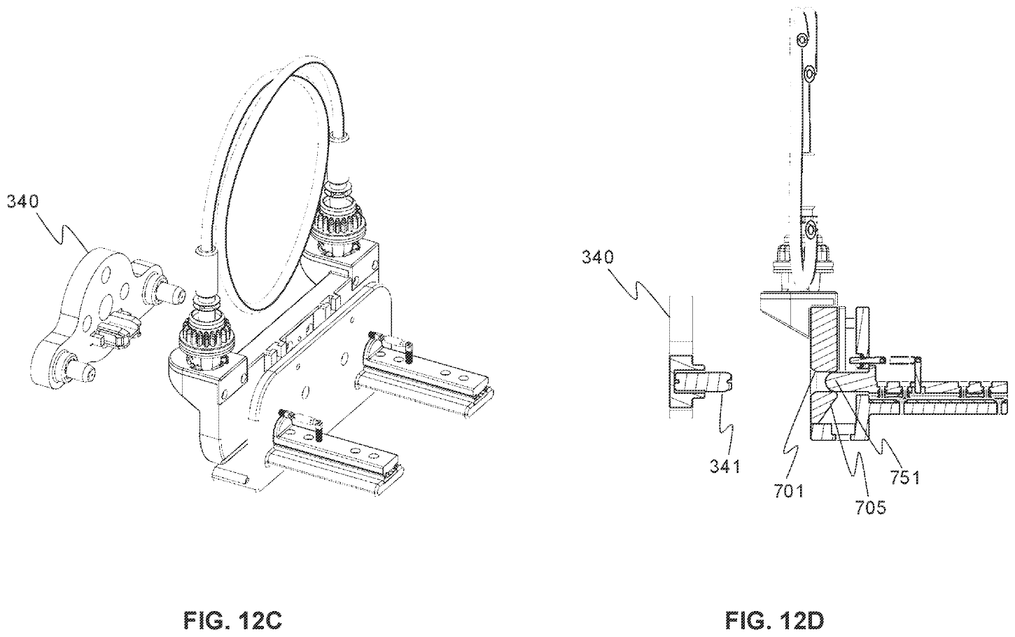

In some embodiments, the rigid cartridge includes at least one rigid surface (e.g., a rigid cartridge). In some embodiments, the rigid cartridge includes a plurality of rigid walls that define a fixed interior. Moreover, the plurality of rigid walls includes a plurality of rigid side walls. For instance, Specifically, rigid cartridge 300 includes rigid walls 1110 that define a fixed interior. In some embodiments, fixed interior of rigid walls 1110 is utilized to store a portion of a cellular engineering target 2 manufactured by biological foundry system (e.g., rigid cartridge of FIG. 7B). In alternative embodiments, the fixed interior of the rigid walls accommodates a soft body bioreactor that accommodates the cellular engineering target, such as soft body bioreactor 430 of FIG. 12. In this way, the rigid side walls protect the fixed interior of the rigid cartridge (e.g., from contamination and/or external forces, such as an articulated handling robot). Edges formed by the rigid side walls includes a radius of curvature greater than zero, which provided rounded or chamfered edges, allowing for an articulated handing robot to grasp the exterior of the rigid walls of the rigid cartridge device when the articulated handing robot is not accurate and/or precise in locating the rigid cartridge device. Furthermore, in some embodiments, the rigid cartridge 300 includes substantially planar upper rigid wall 710-1 that is connected to an upper edge portion of each rigid side wall 720. The substantially planar upper rigid wall includes apertures (e.g., apertures 302 of FIG. 7A). Each respective aperture 302 is configured to receive and fixedly engage a corresponding connector (e.g., connectors 320 of FIG. 7C), which allows for communication with the interior of the rigid cartridge. For instance, each respective connector provides communication with a corresponding port in a first plurality of ports of the soft body bioreactor. However, the present disclosure is not limited thereto. In some embodiments, each respective connector provides communication with a corresponding port in a second plurality of ports of a manifold configured to generate a pressure gradient between the interior of the rigid cartridge and a portion of the manifold, such as to provide and/or receive a medium to the rigid cartridge.

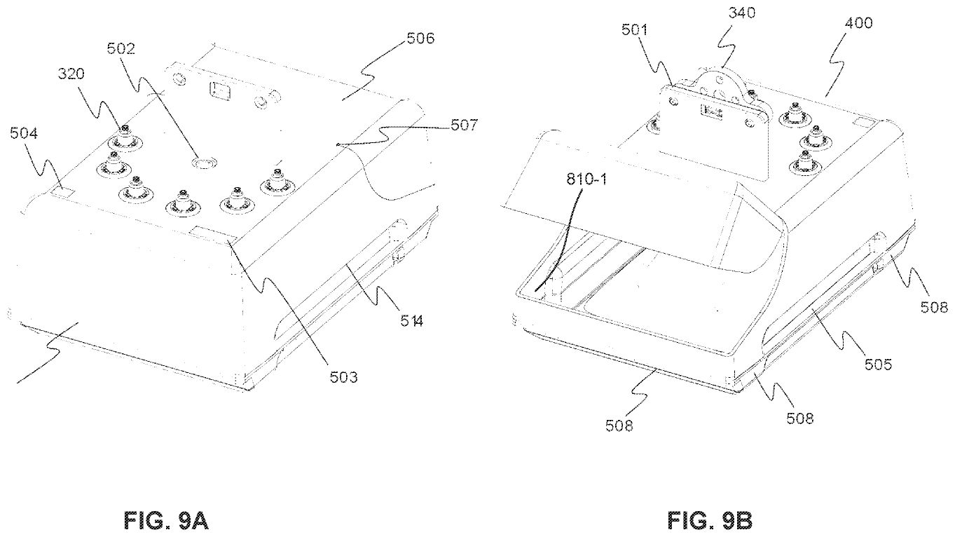

At least one interior surface 810 of a rigid side wall in the plurality of side walls includes first mating mechanism 510 that configured to engage corresponding second mating mechanism 910 of a docking device and/or a soft body bioreactor. For instance, referring to FIG. 9C, in some embodiments, the rigid cartridge is configured as a hard casing configured to accommodate soft body bioreactor 403. In some embodiments, the rigid cartridge has a structure is transportable by a robot, such as by providing one or more rigid mounting surfaces for connectors 320 that the robot interacts with in a repeatable manner. However, in some such embodiments, this ability to transport the rigid cartridge in space requires that a medium (e.g., fluid accommodated by soft body bioreactor 403) to be physically stable or substantially stable.

In some embodiments, the first mating mechanism of the cartridge and the second mating mechanism of the docking device and/or the soft body bioreactor includes one or more snap-fit mating mechanisms and/or one or more clamp mating mechanisms. For instance, in some embodiments, the second mating mechanism of the docking device and/or the soft body bioreactor includes one or more rigid rods 509 at a first end portion the second mating mechanism of the docking device and/or the soft body bioreactor and a second end portion the second mating mechanism of the docking device and/or the soft body bioreactor, such as opposing end portions the second mating mechanism of the docking device and/or the soft body bioreactor. In some embodiments, the one or more rigid rods is configured to be accommodated by a corresponding groove (e.g., mating mechanism 510). In some embodiments, the second mating mechanism of the docking device and/or the soft body bioreactor is configured to compensate (e.g., dampen) for any non-upward forces the docking device and/or the soft body bioreactor is subject to when coupled with the rigid cartridge. However, the present disclosure is not limited thereto.

In some embodiments, the rigid cartridge further includes a lower end portion that includes opening 511. In some embodiments, the opening is configured to accommodate a port of fluidic container. In some embodiments, the opening is configured to accommodate, at least a part, a portion of a docking device, such as a corresponding second mating mechanism of the docking device. In some embodiments, the lower end portion of the cartridge includes the opening 511 that is configured to allow for physical contact between a lower surface of the soft body bioreactor 403 and an upper surface of a docking device and/or an upper surface of a rocking device, such as a rocking docking device. In some embodiments, the opening is configured to provide access for one or more sensors to interface with a surface and/or interior volume of the soft body bioreactor. In some embodiments, the opening is configured for controlling experimental conditions, like temperature, pressure, humidity, electric charge, orientation, or a combination thereof.

In some embodiments, the corresponding second locking mechanism includes a rotating fastener configured to pivot between a first position that restricts a movement of the rigid cartridge and a second position that allows for movement of the rigid cartridge. In some embodiments, the first locking mechanism and the corresponding second locking mechanism collectively form an undercarriage of the apparatus.

In some embodiments, the first mating mechanism includes one or more magnets, one or more fasteners, one or more snap-fittings, one or more press-fittings, one or more adhesives, one or more grooves, one or more holes, one or more openings, one or more protrusions, one or more pins, one or more wedges, one or more indica, one or more snap mechanisms, one or more ramps, one or more springs, or a combination thereof. In some embodiments, each mating mechanism protrudes from the substantially upper planar surface of the at least one rigid wall of the rigid housing.

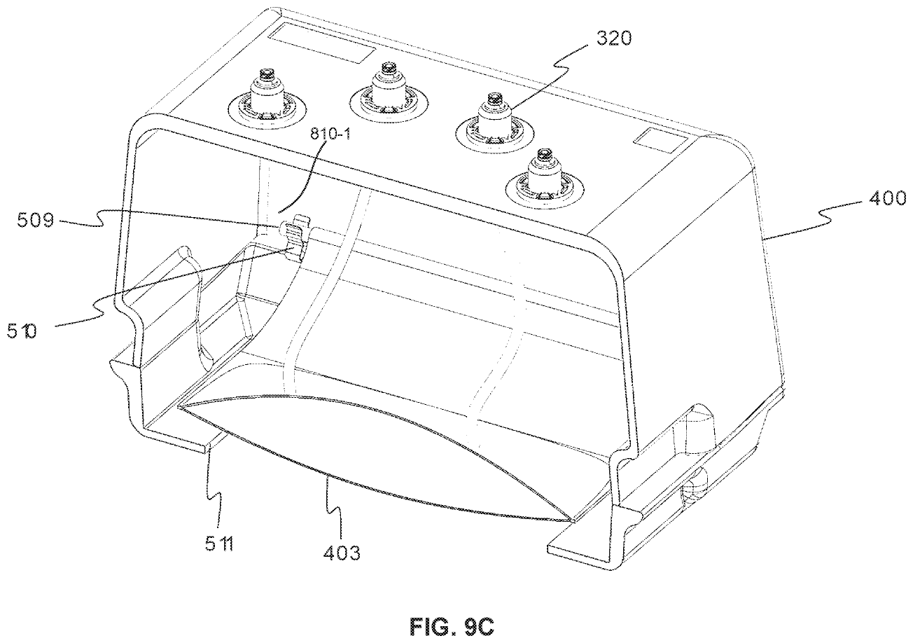

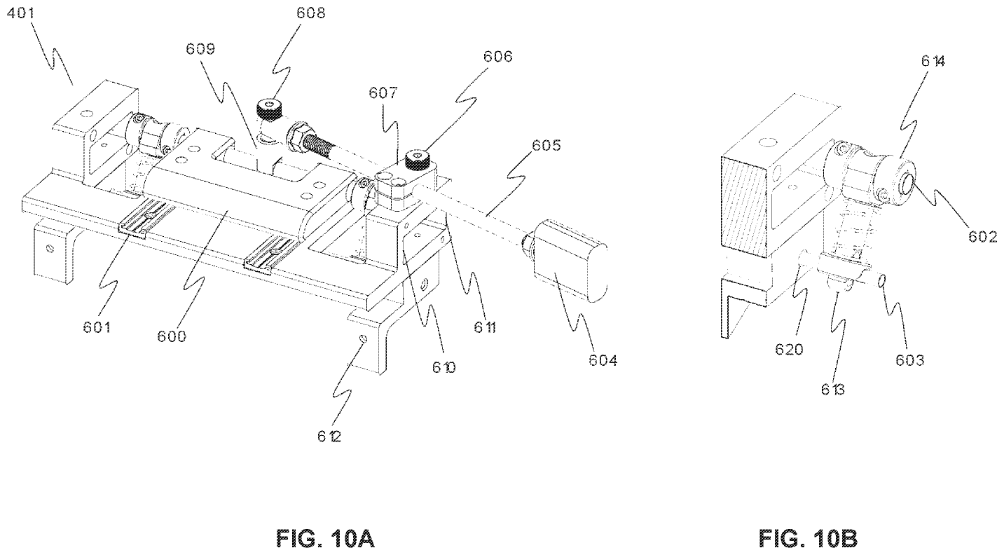

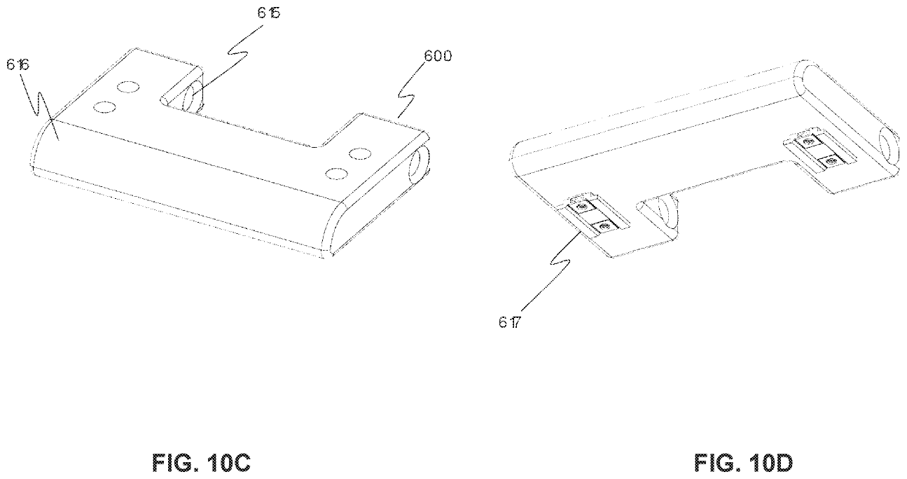

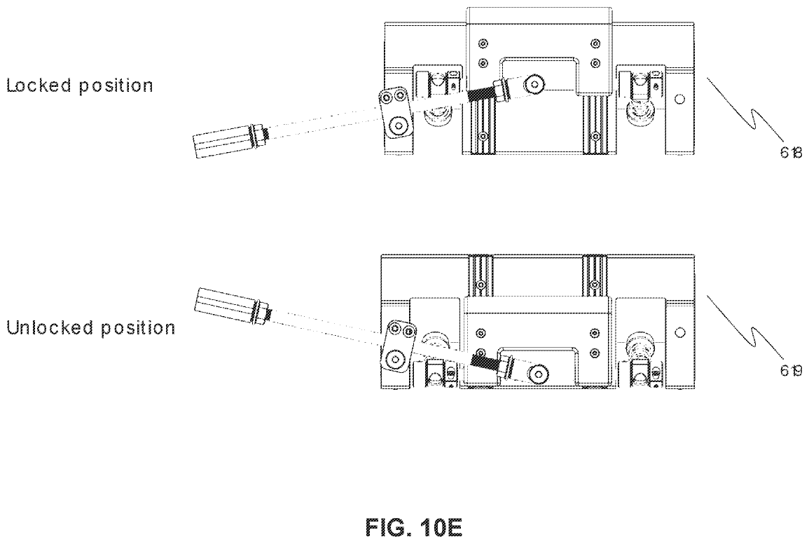

For instance, in some embodiments, the first mating mechanism is configured for a robotic system to interface with. Moreover, in some embodiments, the first mating mechanism allows for a respective docking device to keep the rigid cartridge at a position when coupled with a corresponding second mating mechanism of the docking device due to gravity, such as by including the one or more wedges and/or one or more chamfer edges. However, the present disclosure in not limited thereto. Moreover, in some embodiments, the first mating mechanism is configured to change states (e.g., change configurations from a first locked state FIG. 10E to a second unlocked state of FIG. 10E), such as when a first force is applied to the first mating mechanism, in which the first force is perpendicular or substantially perpendicular to gravity and/or a second force is applied to the first mating mechanism, in which the second force is parallel to gravity. Moreover, in some embodiments, the first mating mechanism allows for requiring high accuracy and precision when coupling the docking device and the rigid cartridge through the first and second mating mechanisms. Moreover, in some such embodiments, the first mating mechanism allows for a high rigidity, such that the first mating mechanism is not tolerant to misalignments when coupling with a corresponding second mating mechanism of a docking device.

In some embodiments, the first degree of freedom is parallel or substantially parallel to gravity. In some embodiments, the first degree of freedom is not parallel or substantially parallel to gravity. In some embodiments, the first mating mechanism and the second mating mechanism is configured to restrict a translational degree of freedom between the rigid cartridge and the docking device and/or a rotational degree of freedom between the rigid cartridge and the docking device, two translational degrees of freedom between the rigid cartridge and the docking device and/or two rotational degrees of freedom between the rigid cartridge and the docking device, or three translational degrees of freedom between the rigid cartridge and the docking device and/or three rotational degrees of freedom between the rigid cartridge and the docking device. For instance, in some embodiments, the translational degree of freedom is a distance between a first axis of the rigid cartridge and a second axis the docking device. In some embodiments, the rotational degree of freedom is an angle between the first axis of the rigid cartridge and the second axis the docking device. However, the present disclosure is not limited thereto.

Accordingly, the first mating mechanism and the corresponding second mating mechanism allow for removably coupling the rigid cartridge to the docking device and/or soft body bioreactor when the first mating mechanism interfaces with the corresponding second mating mechanism to restrict a movement of the rigid cartridge to at least one degree of freedom.

In some embodiments, the at least one interior surface is in the pair of opposing interior surfaces of two rigid side walls in the plurality of rigid side walls that each fourth second mating mechanism of a soft body bioreactor accommodated by the rigid cartridge, thereby removably coupling a pair of opposing end portions of the soft body bioreactor with the rigid cartridge.

In some embodiments, a first internal diameter at an upper end portion of a respective connector is less than a second internal diameter at a lower end portion of the respective connector. This allows the medium distribution system and/or articulated handling robot to engage the connector easily given the defined curvature.

In some embodiments, at least one rigid wall in the plurality of rigid walls further includes a gate mechanism configured to provide access to the fixed internal cavity of the rigid housing. In some embodiments, a length of the gate mechanism is greater than or equal to a cross-sectional area of the soft body bioreactor 403, which allows for the soft body bioreactor to be received by the interior of the rigid cartridge from at least one orientation of the soft body bioreactor.

In some embodiments, the apparatus further includes a corresponding cap for each respective connector in the plurality of connectors, in which the corresponding cap encompasses the respective connector.

In some embodiments, an interior angle formed between a substantially planar upper rigid wall and each rigid sidewall in the plurality of rigid side walls is greater than or equal to about 90 degrees (°), greater than or equal to about 100°, greater than or equal to about 110°, greater than or equal to about 120°, greater than or equal to about 130°, greater than or equal to about 140°, or greater than or equal to about 150°. In some embodiments, an interior angle formed between a substantially planar upper rigid wall and each rigid sidewall in the plurality of rigid side walls is no more than about 90°, no more than about 100°, no more than about 110°, no more than about 120°, no more than about 130°, no more than about 140°, or no more than about 150°.

In some embodiments, an exterior surface of the rigid cartridge includes one or more identifiers indica configured to uniquely identify the rigid cartridge. Moreover, in some embodiments, an exterior surface of the rigid cartridge includes one or more orientation indica configured to identify an orientation of the rigid cartridge. For instance, in some embodiments, the one or more identifiers indica includes with a corresponding barcode that communicate identity information (ID) and/or spatial positioning information (e.g., addresses 104 of FIG. 2) associated with the rigid cartridge. In some embodiments, one or more cameras or vision systems is disposed on stationary or mobile assemblies (e.g., on a gripper device of the medium distribution system, in the modules, on the frame of the modular biological foundry system, etc.), which allows for remote observation of the rigid cartridge. With this barcode, each cellular engineering target and/or each step of the process of manufacture of cellular engineering targets associated with a rigid cartridge is automatically tracked by the medium distribution system and/or computer system 100. Therefore, the medium distribution system automatically maintains a full, real-time audit trail for each cellular engineering target. Each step in the process, one or more numerical values (or measurements) associated with the manufacturing step (e.g., temperature, concentration, elapsed time, velocity, etc.), and/or a time stamp is automatically logged and communicated via communications network to the computer system 100. In some embodiments, this real-time, automatic tracking enables the modular biological foundry system to guarantee that there is never confusion between multiple cellular engineering targets. Accordingly, the medium distribution system described herein can manufacture multiple cellular engineering targets in parallel (at the same time), because each of the cellular engineering targets is completely tracked by the barcode tags and by the vision system of modular biological foundry system. In some embodiments, the one or more identifier indica includes one or more radio-frequency tags, one or more magnetic tags, one or more tags that do not rely on video data, or a combination thereof. Moreover, in some embodiments, the one or more indicia is utilized to determine an orientation and/or position of the rigid cartridge within the modular biological foundry system. For instance, in some embodiments, the one or more indica includes a bar code tag 503 with unique information associated with the rigid cartridge, and/or a reference tag 504 which is used by the articulated handling robot to register the gripper's location relative to the cartridge, such as via visual recognition with a camera mounted on the articulated handling robot and/or in the module frame.

Referring briefly to FIGS. 7A, 7B, and 7B, in various embodiments, the present disclosure provides a rigid cartridge, such as rigid cartridge 300. In some embodiments, the rigid cartridge 300 is configured to interface (e.g., removable couple) with a membrane-based container 304, and the corresponding docking device 310. However, the present disclosure is not limited thereto. The docking device instrument utilized by a modular biological foundry system combines exceptional positional accuracy with an improved tolerance to dimensional errors.

In some embodiments, a container (e.g., membrane-based container 304) is coupled to the lower end portion of the cartridge 300, such as by interfacing a third mating mechanism of the container with the first mating mechanism of the rigid cartridge. For instance, in some embodiments, the container includes one or more threats at an upper end portion of the container and the first mating mechanism of the container includes one or more grooves at the lower end portion of the cartridge. However, the present disclosure is not limited thereto. For instance, in some embodiments, the first mating mechanisms and/or second mating mechanism includes one or more mating mechanism on the lower end portion configured to guide a position of the container, such as one or more slotted holes 303 that center the position of the container and mate with protrusions mating mechanism, such as one or more pins, one or more wedges, or other locating mating mechanisms 312 located on the docking device 310. However, the present disclosure is not limited thereto.

In some embodiments, the membrane-based container is configured to be moved between locations with the help of a motorized electromechanical device, such as articulated handling robot, with an appropriate gripping device (e.g., gripping device 340 of FIGS. 7G through 7I). In some embodiments, the gripping device of the articulated handling robot couples with the container using one or more protrusions, such as one or more pins 341 that couples with corresponding mating mechanisms, such as one or more slotted holes or grooves 301 in the cartridge. In some such embodiments, this structure of the gripping device and the first mating mechanism of the cartridge allows for repeatable placement of the container on the lower end portion of the cartridge and/or an apparatus, such as on protrusions 312 of the docking device 310. In some embodiments, after the gripping device 340 places the cartridge on the docking device, the articulated handling robot gripping device disengages from a surface of the container allowing the spring-loaded 317 z-axes mating mechanism 313 to move into the slotted holes, which ensures there is no unintended motion in any axis. For instance, in some embodiments, the second mating mechanism of the docking device and/or the soft body bioreactor includes one or more springs that are configured to extend from a compressed state to engage with the first mating mechanism of the rigid cartridge. However, the present disclosure is not limited thereto.

Referring briefly to FIGS. 7D through 7F, an exemplary docking device for coupling the rigid cartridge in all axes (e.g., all three translational degrees of free and all three rotational degrees of freedom are restricted between the cartridge and the docking device) is provided in accordance with some embodiments. In some embodiments, the first mating mechanism includes protrusion-slot 312, which provide a datum feature for precise location of the cartridge on the docking device, which prevents or substantially retards all direction of motion except the vertical direction (e.g., parallel or substantially parallel to gravity), as defined by the axis perpendicular to the cartridge upper end portion plane 350. In some embodiments, the first mating mechanism includes one or more pins, one or more ramps, one or more wedges, one or more snaps, one or more fasteners, other mating mechanisms, or a combination thereof.

For instance, in some embodiments, the second mating mechanism includes spring loaded mechanism 317, which is configured to couple with a first mating mechanism of the cartridge once the cartridge has been disposed or positioned adjacent to the docking device. In some embodiments, the second mating mechanism is configured to prevent motion of the rigid cartridge in the direction (e.g., parallel or substantially parallel to gravity). In some embodiments, for the container coupled to the rigid cartridge to be moved in and out of the docking device, the z-axis locking mating mechanism 314 moves between the lock and unlock positions. In some embodiments, this movement is provided by mounting the z-axis locking mating mechanisms on a rail 316 using one or more carriages 315. In some embodiments, the rail is replaced by T-slot groove or one or more slides. In some embodiments, to maintain clearance for the bioreactor 304, the docking device is partitioned into mirrored halves 311 that is mounted to a mounting plate 319, such as by using various types of fasteners. In some embodiments, the cartridge is secured on the docking device only by the locating mating mechanisms 312. In some such embodiments, the docking device does not use z-axis locking mating mechanism when gravity is enough to keep the cartridge in place in the docking device.

In some embodiments, once the container is loaded onto the docking device the connections is made with any of the connectors 320. In some embodiments, the connector assembly 320 includes one end of an air-tight connector, such as a female Luer connector 323, which when mated with a male Luer connector 331 produces an air-tight connection. In some embodiments, the connector assembly 320 consists of one end of an air-tight connector. In some embodiments, the tubes from the connector 320 are fed into a sterilized container (e.g., manually fed), such as the sterile fluid path 304 container. In some embodiments, the male Luer connector is screwed on to the female luer connector, such as via a rotating tool, such as a syringe gripper 100 with a compatible luer connector adapter, similar to a tube holder (e.g., tube holder 330 of FIG. 3B). Additionally, in some embodiments, to have repeatable and reliable connections, one or more locating mating mechanisms such as chamfered extrusions 332 from the tube holder is used, as well as mating mechanism on the gripper to enable visual recognition with a camera and/or depth sensors, edge finders, contact sensors, or a combination thereof.

Referring briefly to FIGS. 8A through 8C, an exemplary rigid cartridge 400 is provided in accordance with some embodiments. In some embodiments, the rigid cartridge is placed on a rocking docking device 402. In some embodiments, one or more locking mating mechanisms 401 are disposed on the rocking docking device. In some embodiments, the one or more locking mating mechanism is actuated by the articulated handling robot to secure the cartridge on the bioreactor. In some embodiments, the cartridge is a hard casing for a soft body bioreactor 403 that the articulated handling robot can transport and provides a rigid mounting surface for connectors 320 that the articulated handling robot can interact with in a repeatable manner. In some embodiments, the locking mating mechanisms 401 are electrically controlled, via solenoids or motors for example, or is passively engaging as the cartridge is positioned in place by the articulated handling robot, such as bi-stable locking mechanisms, or spring loaded mating mechanisms (e.g., as described in FIGS. 7D through 7F and/or FIGS. 7G through 7I).

Referring briefly to FIGS. 9A and 9B, in some embodiments, the rigid cartridge 400 is a hard casing for a soft body bioreactor 403 that the articulated handling robot can transport. In some embodiments, the rigid cartridge provides a rigid mounting surface for connectors 320 that the articulated handling robot can interact with in a repeatable manner.

In some embodiments, the cartridge is picked up by the gripper from the gripper mating mechanism 501, which is located vertically above the soft body bioreactor's center of mass to prevent instability of the cartridge when being transported by the gripper. In some embodiments, when the articulated handling robot places the cartridge on the rocking docking device, the cartridge self-centers itself with sufficient lead-in mating mechanism 508 such as one or more chamfers, one or more wedges, one or more slots, one or more dimensional restraints, or a combination thereof. In some embodiments, the self-centering is provided through one or more springs or other elastic members, one or more locating pins, one or more magnets, or a combination thereof. In some embodiments, the self-centering mating mechanisms prevent the lateral movement of the cartridge with respect to the rocking docking device.

In some embodiments, the cartridge includes one or more ledges 505 that the locking mating mechanisms 401 disposed on the rocking docking device come in contact with to prevent vertical movement of the cartridge that results from the rocking of the bioreactor or the articulated handling robot interacting with the connectors on the cartridge.

In some embodiments, the cartridge includes one or more connectors 320 that allows the articulated handling robot to connect tubing to the soft body bioreactor, such as tubing for transferring gas, media or other liquids. In some embodiments, the connector bases are mounted to the external surface of the cartridge using a fastening method such as, but not limited to, ultrasonic welding, bonding, interference snaps, or fasteners.

In some embodiments, electronic connector 502 allows the articulated handling robot to connect the cartridge electrically to the equipment, for example to provide power for temperature control or to power an active system or sensors, as well as communication channels for data exchange. In some embodiments, the power system includes one custom electrical connection or multiple separate connections. In some embodiments, the connector 502 includes one or more fiber optic connections for one or more sensors such as pH and/or oxygen sensors, or individually separate connectors.

Additionally, in some embodiments, the cartridge includes one or more handle mechanism 514 to be used by one or more users carrying the cartridge. In some embodiments, the one or more handle mechanism include user mounted handles.

In some embodiments, such as in the event that the one or more users requires access or a visual inspection of the soft body bioreactor during the experimental process, the cartridge back door 506 is raisable. In some embodiments, the door rotates around a hinge 507. In some embodiments, the visual access is provided using a sliding door and/or transparent window.

Referring to FIGS. 10A and 10E, a bi-stable locking mechanism 401 with an extended handle 604 that is actuatable by the articulated handling robot is provided. In some embodiments, the locking mechanism is attached to each side of the rocking docking device 402 and is used to constrain the position of the cartridge 400 when placed on the rocking docking device. In some embodiments, the mechanism is attached to the bioreactor tray with fasteners through mating mechanism 612.

In some embodiments, the articulated handling robot pushes on the handle 604, and this force is translated into the mechanism through handle rod 605. In some embodiments, the shaft collar pivot 607 constrains the handle rod axially and rotationally. In some embodiments, through the shaft collar pivot, the handle rod rotates about primary pivot 606. In some embodiments, the end of the handle rod pushes on secondary pivot 608, which is held vertical since the rod is constrained rotationally. In some embodiments, the secondary pivot is attached to a rod end 609 that is able to slide axially along the primary rod 602 and translates lateral force through the approximate center of the primary rod, which is fixed rotationally and axially in latch 600 by mating mechanism 615. In some embodiments, the lateral force is translated into the linear motion of latch 600, which slides along linear rails 601 on carriages 617.

In some embodiments, in order to create two stable states of the mechanism, locked 618 and unlocked 619, one or more spring-loaded plungers 613 apply the resistive force to the lateral motion of the primary rod 602 and, therefore, the latch 600. In some embodiments, the resistive force is provided through one or more opposing magnets, one or more leaf springs, one or more pneumatic springs, or a combination thereof.

In some embodiments, the plungers are supported at one end by the primary rod, and at the other end by pivoting around secondary rod 603. In some embodiments, the secondary rod is fixed rotationally and axially by mating mechanism 620. In some embodiments, the position of the plungers are constrained axially along the primary rod by mating mechanism 614. In some embodiments, the mating mechanisms 614 are shaft collars on the primary rod. In some embodiments, the mating mechanism 614 are shaft collars on the secondary rod, grooves in the primary and/or secondary rods, flanges, other positioning mating mechanism used on shafts, or a combination thereof.

In some embodiments, hard stops 610 and 611 set the stroke distance of the primary rod 602, which in turn sets how far the latch 600 travels. In some embodiments, when the articulated handling robot actuates the mechanism such that the primary rod is in contact with hard stop 610, the latch is in the locked position 618, and vertically constrains the cartridge 400 in the rocking docking device 402. In some embodiments, when the articulated handling robot actuates the mechanism such that the primary rod is in contact with hard stop 611, the latch is in the unlocked position 619, the latch remains out of the cartridge's path into and out of the bioreactor.

In some embodiments, the articulated handling robot manually unlocks and/or locks the cartridge as needed. For instance, in some embodiments, the cartridge uses automatic locking and manual unlocking. In some embodiments, when the latch is in the locked position 618, the cartridge 400 is pushed down into the bioreactor tray, which pushes the latch 600 out of the way. In some embodiments, when the midpoint of the bi-stable mechanism is not reached, the latch snaps back into the locked position. In some embodiments, when the process includes the cartridge being removed, the articulated handling robot manually moves the handle of the mechanism into the unlocked position 619. In some embodiments, the latch has appropriate lead-in 616 on an upper end portion thereof for the cartridge to push the latches out of the way when coming from above. In another embodiment, a motorized system on the tray moves the bi-stable mechanism in the un-locked state and back in the locking state, by using motors, solenoids or similar actuation means.

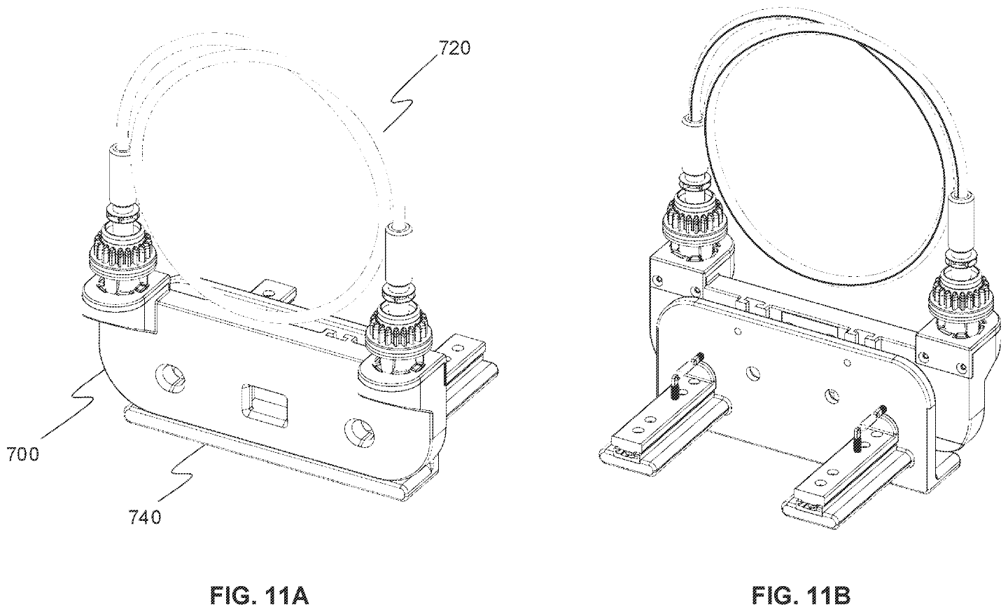

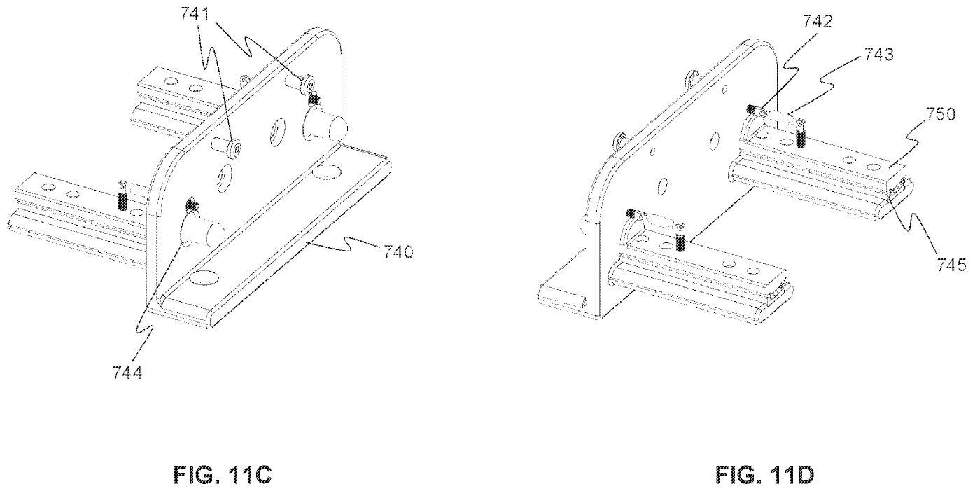

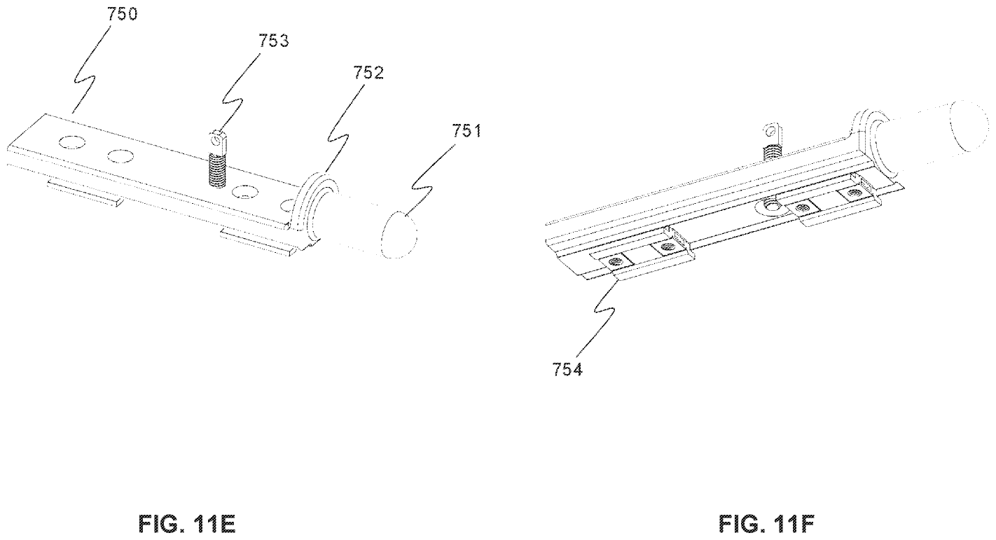

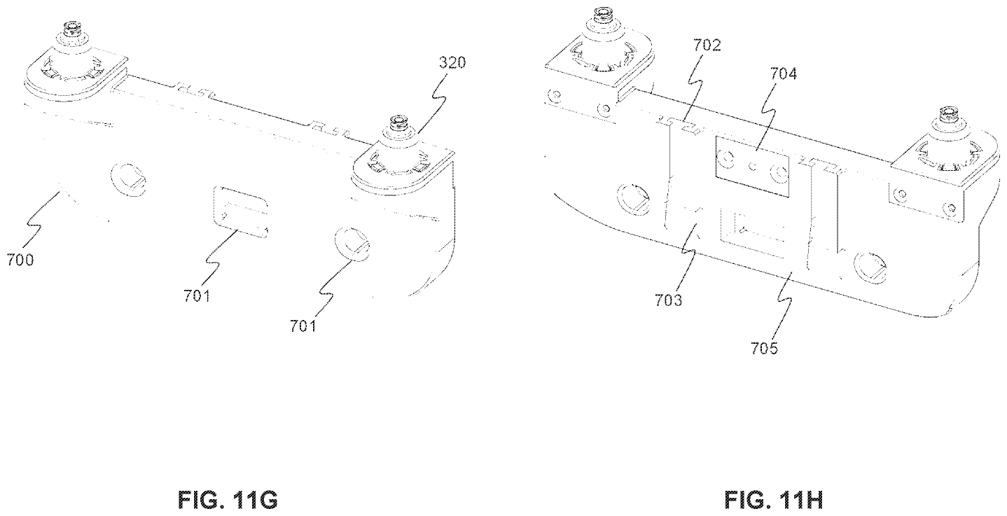

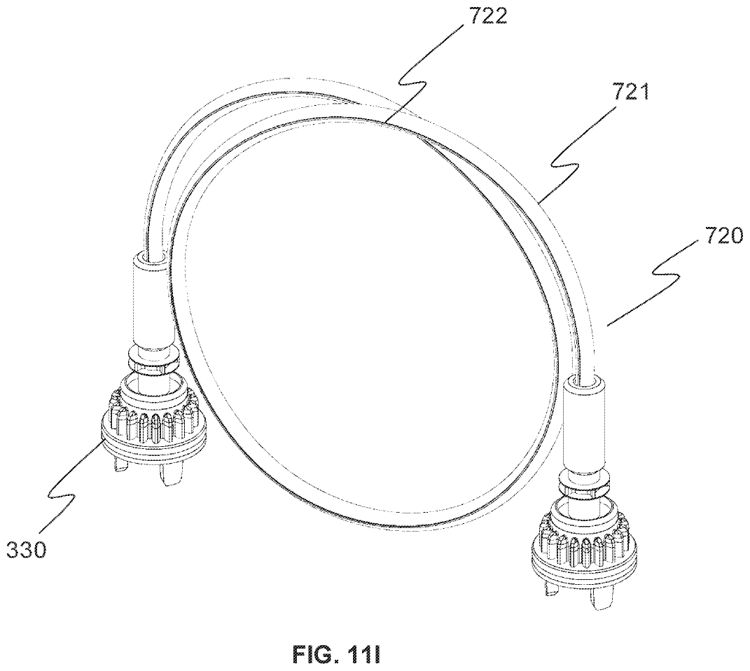

Referring to FIGS. 11A and 11B, an embodiment of an apparatus including a rigid cartridge 700 coupled to a docking device 740 is provided. In some embodiments, the cartridge acts as a vehicle for the articulated handling robot to transport a portion of a manifold 720 reliably within a clean-room environment.

In some embodiments, the rigid cartridge includes a substantially planar upper rigid wall that connected to an upper edge portion of each rigid side wall in the plurality of rigid side walls. Furthermore, the substantially planar upper rigid wall includes one or more apertures. In some embodiments, each respective aperture in the one or more apertures is configured to receive and fixedly engage a corresponding connector in one or more connectors. In some embodiments, the corresponding connector is permanently coupled to the rigid cartridge. However, the present disclosure is not limited thereto. Moreover, each respective connector in the one or more connectors interfaces with a corresponding portion of a fixed interior of the rigid cartridge.

In some embodiments, the one or more connectors includes a fluidic connector and/or an electrical connector. In some embodiments, the electrical connector of the rigid cartridge includes a pressure control mechanism (e.g., valve), a pH sensor, a dissolved oxygen sensor, a temperature sensor, a flow rate sensor, a mass sensor, or a combination thereof. In some embodiments, the fluidic connector includes a pressure control mechanism, an inlet port, a sampling port, an outlet port, or a combination thereof.

In some embodiments, the fluidic connector is in communication with a manifold configured to generate a pressure gradient between a portion of the rigid cartridge and an interior of the manifold. In some embodiments, the fluidic connector of the rigid cartridge includes a pressure control mechanism, an inlet port, a sampling port, an outlet port, or a combination thereof. In some embodiments, the fluidic connector includes a valve configured to control a flow of a fluid through a corresponding fluidic port.

Referring to FIGS. 11C and 11D, an embodiment of the transfer cartridge nest 740 is provided. In some embodiments, the docking device has locating mating mechanisms 741 used to place the rigid cartridge 700 on the docking device in a repeatable position. In some embodiments, the docking device has openings 744 through which the latches 750 slide through. In some embodiments, the latches slide over linear rails or slides 745, and are loaded with an elastic member 743. In some embodiments, the elastic member has an anchor point 742 on the docking device to keep the latch biased to push against the back of the docking device, protruding through the openings with a given preload force. In some embodiments, the elastic members include one or more extension springs, one or more opposing magnets, one or more weights, one or more leaf springs, one or more pneumatic springs, or a combination thereof.

Referring to FIGS. 11E and 11F, an embodiment of the transfer cartridge nest latches 750 is provided. In some embodiments, the latches are mounted on linear rail carriages 754, that slide on linear rails 745 in the docking device. In some embodiments, the protruding end of the latch includes a pin-like rounded or chamfered end 751 that fit into the gripper mating mechanisms 701 of the cartridge, a hard stop 752 for stopping against the back of the docking device, and an anchor point 753 for the elastic member 743 that is also attached to the docking device at the other end.

Referring to FIGS. 11G and 11E, an embodiment of the rigid cartridge 700 without a portion of a manifold disposed is provided. In some embodiments, the cartridge includes one or more connectors 320, in which each connector in the one or more connectors is configured as a port (e.g., a docking point) for a corresponding connector of the manifold. Accordingly, the connectors allow for electrical and/or fluidic communication between at least the interior volume of the cartridge and the manifold. However, the present disclosure is not limited thereto.

In some embodiments, the cartridge also has mating mechanisms 701 that a articulated handling robot could use to dispose itself into and transport the cartridge. In some embodiments, the back of the cartridge includes one or more slots 702 for locating mating mechanism 741 on the docking device to insert into. The articulated handling robot would bring the cartridge to above the docking device, then lower onto the locating mating mechanism and back out. In some embodiments, when the cartridge is disposed on the docking device, the slots interact with the locating mating mechanism 741 to counteract forces perpendicular to the front face of the cartridge, such as when the articulated handling robot gripper pulls out of the mating mechanism 701. In some embodiments, the lead-in 703 into the slots 702, helps to center the locating mating mechanism as the articulated handling robot lowers the cartridge, allowing the correct loading of the cartridge also in presence of mis-alignment tolerances due to articulated handling robot accuracy and repeatability, as well as manufacturing and assembly tolerances. In some embodiments, the large chamfers 705 are used to push the latches 750 on the docking device back, against the resistive force of the elastic members 743, as the gripper lowers the cartridge onto the docking device. In some embodiments, the compartment 704 is used to house a magnet, which assists in controlling the position of the tubing coil assembly 720, so long as the elastic member 722 that constrains the tubing is magnetic.

Referring to FIG. 11I, an embodiment of a tubing assembly of a manifold 720 that controls the shape that the flexible tubing 721 takes is provided. In some embodiments, the tubing is attached to an elastic member 722, with a mating mechanism including one or more adhesive materials (e.g., one or more strips of adhesive), lamination, concentric tubes or clips. In some embodiments, the elastic member is a memory coil, but other means is used such as music or spring wire, rigid plastics or composite fiber material compounds, such as carbon fibers. In some embodiments, since attached, the shape of the elastic member controls the shape of the tubing. In some embodiments, this structure allows for a wide range of flexible tubing to be used. In some embodiments, the tubing ends are inserted and secured into tube holders 330, which screw onto the connectors 320 on the rigid cartridge.

Referring to FIGS. 12A and 12B, an embodiment of the gripping device interacting with the rigid cartridge 700 in the unlocked state is provided. In some embodiments, the articulated handling robot has lowered the cartridge onto the docking device 740. In some embodiments, the pins 341 of the gripper are in line with the rounded ends 751, thus preventing the latch 750 from entering the gripper mating mechanism 701 of the cartridge. In some embodiments, as long as the gripper is fully seated in the cartridge, the gripper can move the cartridge vertically, as constrained by the cartridge slots 702 and the docking device locating mating mechanism 741.

Referring to FIGS. 12C and 12D, an embodiment of the robot gripper device interacting with the rigid cartridge 700 in the locked state is provided. In some embodiments, the articulated handling robot has lowered the cartridge onto the docking device 740, and pulled away, leaving the cartridge on the docking device. In some embodiments, when the gripper pins 341 are no longer occupying the gripper mating mechanism 701 of the cartridge, the latches 750 are pulled into the mating mechanism by the elastic members 743. In some embodiments, the latches have significant lead-in when entering the mating mechanism with the rounded ends 751. In some embodiments, when the latches are inserted in the mating mechanism, the cartridge is fully constrained on the docking device, since the docking device locating mating mechanism 741 in the cartridge slots 702 prevent motion perpendicular to the front face of the cartridge, and the latches in the gripper mating mechanism prevent motion parallel to the front face of the cartridge.

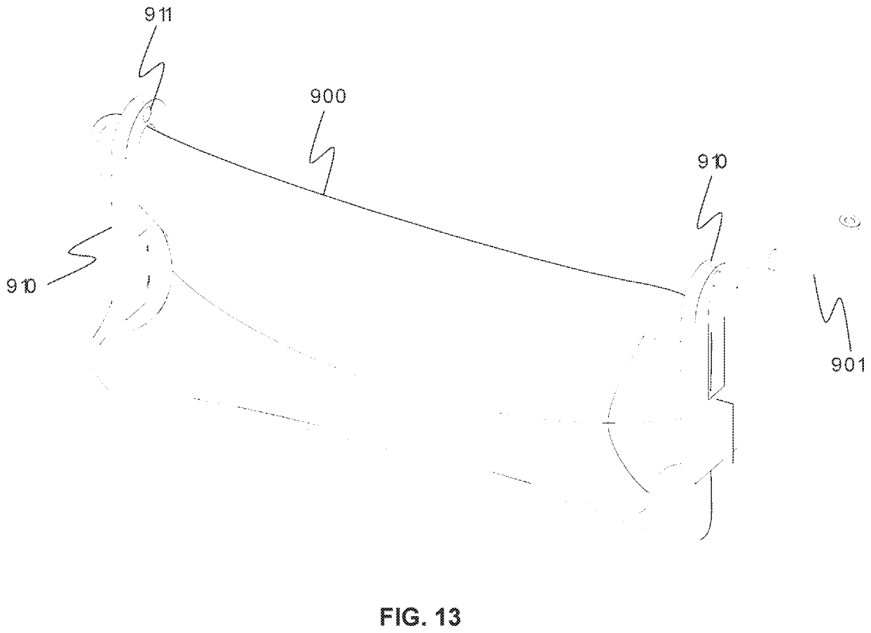

Referring to FIG. 13, an embodiment of a cartridge 900 that stores liquid mediums is provided. In the embodiment shown, the cartridge is a soft body, such as a bog, though this can also be achieved through other embodiments such as a rigid container. In some embodiments, the medium cartridge has tubing 901 that exits the cartridge for the medium to reach downstream applications. In some embodiments, the tube is located on the upper end portion and/or upper edge portion of the cartridge to allow the tube to emptied by gravity. In this way, in some embodiments, the tube remains empty when not in use, and the liquid medium is stored in the bag preventing its degradation in the tube. In some embodiments, this structure is compatible with the dispensing systems and methods described in FIGS. 6A through 6F. In some embodiments, the to use in other assemblies, the cartridge holders 910 secure the cartridge and provide mounting mating mechanism 911 for disposition.

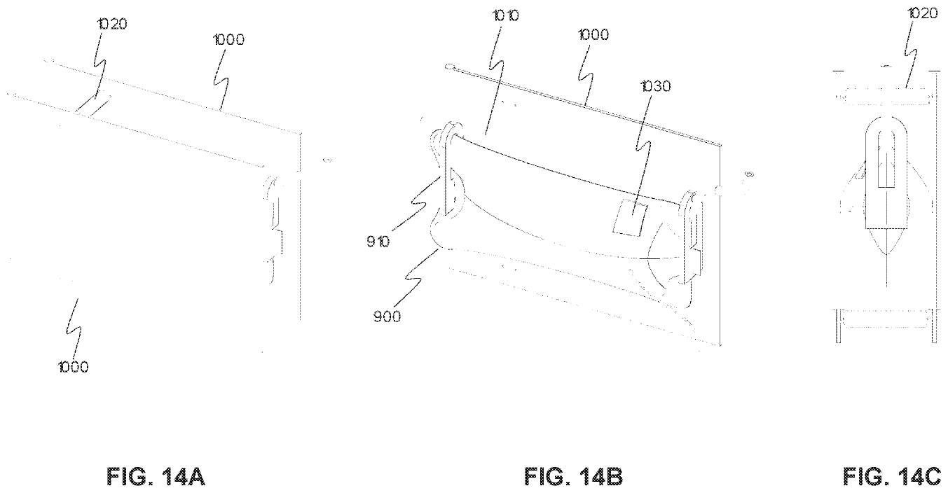

Referring to FIGS. 14A through 14C, an embodiment of a temperature control system for medium storage is provided. In some embodiments, the purpose of the system is to refrigerate the mediums while they are stored for extended periods of time to prevent their degradation.

In some embodiments, conductive plates 1000 are cooled using a device such as a Peltier or liquid-cooled systems, and temperature controller. In some embodiments, the medium cartridge 900 is mounted using the cartridge holders 910 onto the cartridge mounting mating mechanism 1010 so that the cartridge sits in between the plates. In some embodiments, the plates compress the medium cartridge 900 under load from elastic members 1020, such as extension springs, though this can also be achieved in other embodiments such as, but not limited to, compression springs, leaf springs, magnets or pneumatic springs, as well as active means such as motor, pneumatic pistons, linkage systems and dead weights. In some embodiments, the plates cool the medium in the medium cartridge through conduction, and with added air circulation, the cartridge could additionally be cooled through convection. In some embodiments, the compression of the plates helps to maintain good surface contact if the cartridge volume decreases as medium is removed from the cartridge, and to prevent over-compression of the cartridge, the cartridge holders act as a hard stop.

In some embodiments, temperature sensors 1030 measure the temperature of the contents of the medium cartridge 900, and communicate with a temperature controller to adjust the level of cooling to achieve the desired goal temperature. In some embodiments, patch temperature sensors adhered to the outside of the medium cartridge measure the surface temperature of the cartridge, which approximates the internal temperature of the mediums. In some embodiments, the temperature sensors include one or more probe temperature sensors inserted into a pocket in the cartridge or one or more non-contact temperature sensors.

Additionally, in some embodiments, it is necessary to keep track of the weight of the medium that remains in the cartridge as a respective medium is being drawn out pf a corresponding reservoir for downstream applications. In some embodiments, a weighing device is attached to the end of the cartridge mating mechanism 1010, such as a load cell.

In some embodiments, it is necessary to mix the contents of the cartridge to keep the contents a homogeneous mixture and temperature, and a vibrating motor is disposed to the cartridge mating mechanism 1010 to vibrates the cartridge when necessary. In some embodiments, the vibrating motor includes a vibrating mixing plate below the cartridge or utilizes convective mixing by cooling only one side of the cartridge.

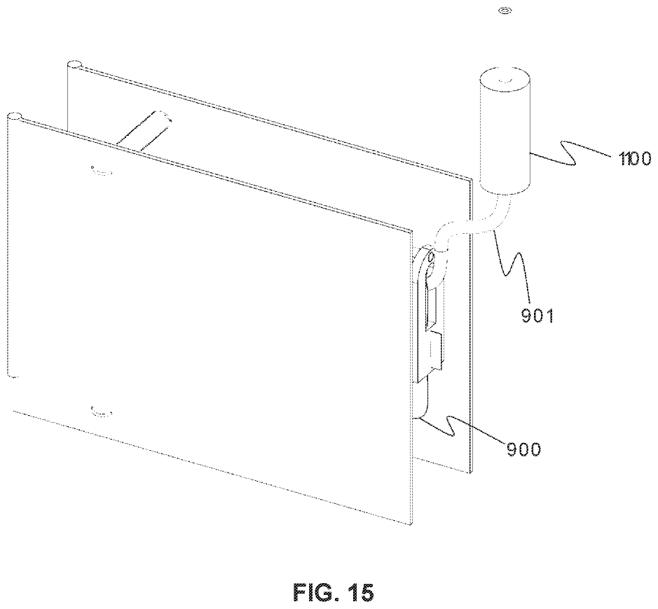

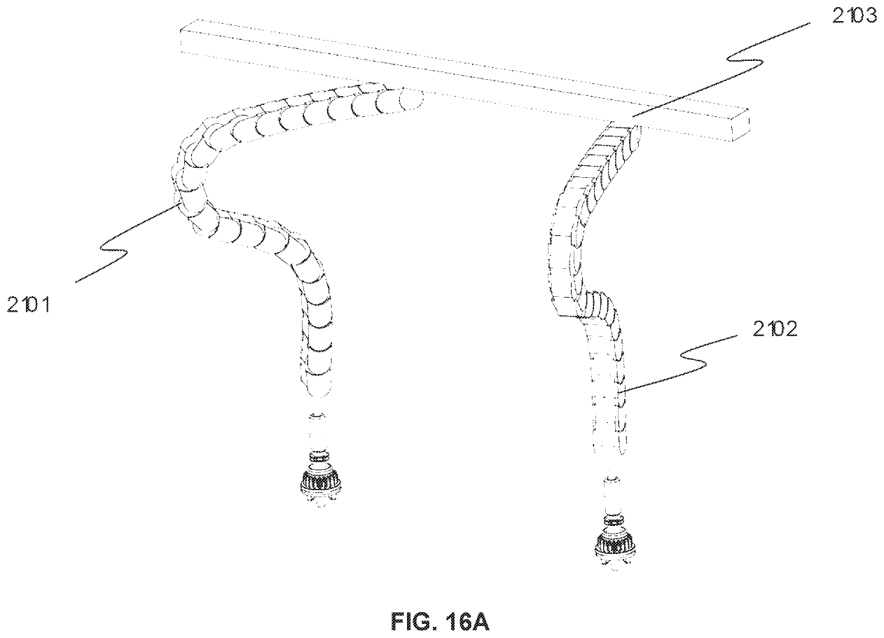

Referring to FIG. 15, an embodiment of the medium temperature control system (e.g., temperature control system of FIG. 10) with an added heating element 1100 is provided. In some embodiments, it is necessary to heat the medium that exits the cartridge 900 through the tubing 901 to a target temperature before the medium reaches the next reservoir or container. In some embodiments, the heating element includes an inline heater or external tubing warmer covering the necessary tube length to achieve the desired temperature.

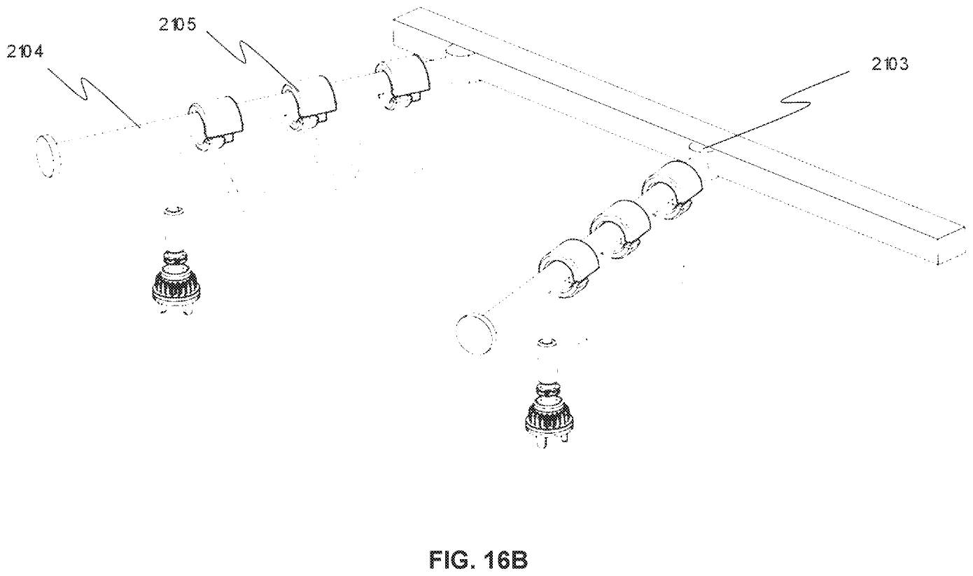

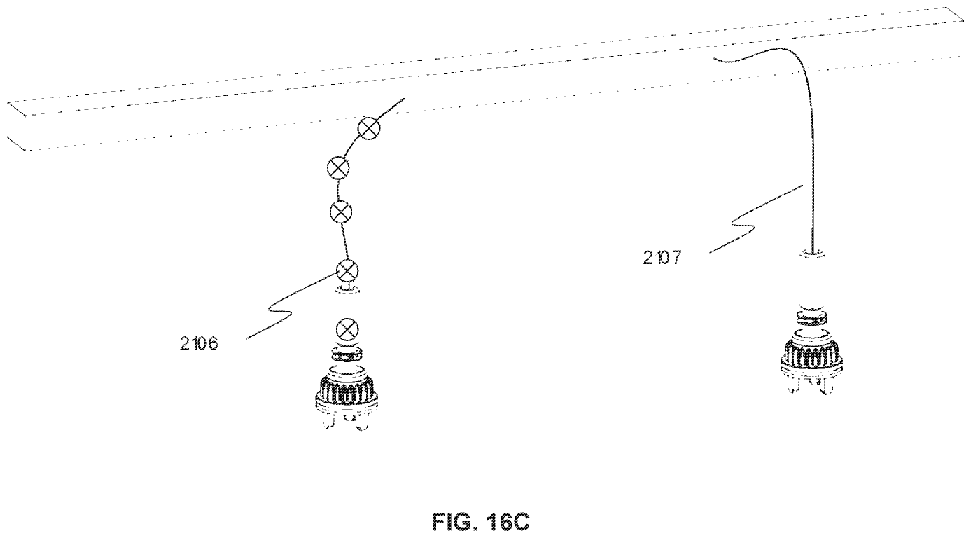

Referring to FIG. 16A, an embodiment of a tubing handling system where the motion of the tubing is constrained by linkages 2101 that reduce one or more degrees of freedom, thus removing uncertainty about the location of the tubing slack and enabling an automated system to manipulate said tubing without interference from the slack is provided. In some embodiments, the tubing handling system prevents this tubing from interfering with the operation of nearby tubing 2102. In some embodiments, the linkages are anchored on a rotating head 2103 providing one or more passive degrees of rotation, such as pitch angle. In some embodiments, the rotating head is attached to an additional passive or active degree of freedom providing linear motion in one or multiple directions (cartesian motion) in order to increase the allowed travel of the connecting tube holder plug while maintaining certainty about the location of the slack.

Referring to FIG. 16B, an embodiment of a tubing handling system where the slack of the tubing is constrained to a single direction by guides 2104, thus removing uncertainty about the location of the tubing slack and enabling an automated system to manipulate said tubing without interference from the slack is provided. In some embodiments, the tubing handling system prevents this tubing from interfering with the operation of nearby tubing. In some embodiments, the tubing slack is attached to the guide using slider rings 2105 or other mechanisms that can move along the guide either freely passively or with a preferential position, for example using gravity to push the sliders toward one end of the rod or by connecting the sliders with springs. In some embodiments, the preferential positions of the sliders along the rod are dictated by notches in the guide rods, by inserting magnets in specific polarity orientation, or by using friction or clutches in the sliders that release motion above a specific pulling force. In some embodiments, the slider rings are motorized and is positioned as desired along the rod. In some embodiments, sensors are used to make the motorized sliders determine and/or generate one or more instructions to move along the rod while keeping a desired amount of tension on the tube, as measured by force sensors in the sliders. In some embodiments, the guides are anchored on a rotating head 2103 providing one or more passive degrees of rotation, such as pitch angle. In some embodiments, the rotating head is attached to an additional passive or active degree of freedom providing linear motion in one or multiple directions (e.g., cartesian motion) in order to increase the allowed travel of the connecting tube holder plug in addition to the direction provided by the guide itself while maintaining certainty about the location of the slack.

Referring to FIG. 16C, an embodiment of a tubing handling system which uses optical means to detect the location of the tubing slack is provided. In some embodiments, one or more optical markers 2106 such as one or more barcodes, one or more marking dots, one or more reflective fiducials, one or more tape are placed on a tubing or a sheath containing such tubing, or a combination thereof. In some embodiments, the one or more optical markers allow the systems and methods of the present disclosure to recognize the position and orientation of the slack portion of the tubing. In some embodiments, the tubing handling system is able to manipulate tubing while avoiding interference from the slack. In another embodiment, a non-contact system capable of sensing three-dimensional position using techniques such as stereo vision, visual recognition, time-of-flight sensors, or fiber Bragg grating is able to detect the location of a tubing or a sheath containing such tubing without using any optical markers 2107. In some embodiments, the base of the tubes is mounted on a pivoting or translating system, or combined with any of the tube handling systems described above.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. An apparatus for manufacture of an engineered cells, the apparatus comprising:
a rigid cartridge comprising a plurality of rigid walls defining a fixed interior; wherein the plurality of rigid walls comprises:
a plurality of rigid side walls, wherein at least one interior surface of a rigid side wall in the plurality of side walls comprises a first mating mechanism configured to engage a corresponding second mating mechanism of a docking device, thereby removably coupling the rigid cartridge to the docking device to restrict a movement of the rigid cartridge to at least one degree of freedom, and
a substantially planar upper rigid wall connected to an upper edge portion of each rigid side wall in the plurality of rigid side walls, wherein the substantially planar upper rigid wall comprises:
one or more apertures, wherein:
each respective aperture in the one or more apertures is configured to receive and fixedly engage a corresponding connector in one or more connectors,
each respective connector in the one or more connectors interfaces with a corresponding portion of a fixed interior of the rigid cartridge.

2. The apparatus of claim 1, wherein the at least one interior surface is in the pair of opposing interior surfaces of two rigid side walls in the plurality of rigid side walls that each fourth second mating mechanism of a soft body bioreactor accommodated by the rigid cartridge, thereby removably coupling a pair of opposing end portions of the soft body bioreactor with the rigid cartridge.

3. The apparatus of claim 1, wherein the one or more connectors comprises a fluidic connector and/or an electrical connector.

4. The apparatus of claim 3, wherein the electrical connector comprises a pressure control mechanism, a pH sensor, a dissolved oxygen sensor, a temperature sensor, a flow rate sensor, a mass sensor, or a combination thereof.

5. The apparatus of claim 2, wherein the fluidic connector comprises a pressure control mechanism, an inlet port, a sampling port, an outlet port, or a combination thereof.

6. The apparatus of claim 2, wherein the fluidic connector is in communication with a manifold configured to generate a pressure gradient between a portion of the rigid cartridge and an interior of the manifold.

7. The apparatus of claim 2, wherein, the fluidic connector comprises a valve configured to control a flow of a fluid through a corresponding fluidic port.

8. The apparatus of claim 2, wherein a first internal diameter at an upper end portion of a respective connector is less than a second internal diameter at a lower end portion of the respective connector.

9. The apparatus of claim 1, wherein at least one rigid wall in the plurality of rigid walls further comprises a gate mechanism configured to provide access to a fixed internal cavity of the rigid housing.

10. The apparatus of claim 1, wherein each mating mechanism protrudes from the substantially upper planar surface of the at least one rigid wall of the rigid housing.

11. The apparatus of claim 10, further comprising a corresponding cap for each respective connector in the plurality of connectors, wherein the corresponding cap encompasses the respective connector.

12. The apparatus of claim 1, wherein the rigid cartridge further comprises a lower end portion comprising an opening configured to accommodating a port of fluidic container.

13. The apparatus of claim 1, wherein the first mating mechanism comprising one or more magnets, one or more fasteners, one or more snap-fittings, one or more press-fittings, one or more adhesives, one or more grooves, one or more holes, one or more openings, one or more protrusions, one or more pins, one or more wedges, one or more indica, one or more snap mechanisms, one or more ramps, one or more springs, or a combination thereof.

14. The apparatus of claim 1, wherein the first degree of freedom is parallel or substantially parallel to gravity.

15. The apparatus of claim 1, wherein the first degree of freedom is not parallel or substantially parallel to gravity.

16. The apparatus of claim 1, wherein an interior angle formed between a substantially planar upper rigid wall and each rigid sidewall in the plurality of rigid side walls is greater than or equal to 90 degrees.

17. The apparatus of claim 1, wherein an exterior surface of the rigid cartridge comprises one or more identifiers indica configured to uniquely identify the rigid cartridge.

18. The apparatus of claim 1, wherein an exterior surface of the rigid cartridge comprises one or more orientation indica configured to identify an orientation of the rigid cartridge.

19. The apparatus of claim 1, wherein the corresponding second locking mechanism comprises a rotating fastener configured to pivot between a first position that restricts a movement of the rigid cartridge and a second position that allows for movement of the rigid cartridge.

20. The apparatus of claim 1, wherein the first locking mechanism and the corresponding second locking mechanism collectively form an undercarriage of the apparatus.
